(19) United States
(12) Patent Application Publication
(10) Pub. No.: US 2011/0243921 A1
(43) Pub. Date: Oct. 6, 2011

(54) PREVENTION AND TREATMENT OF CUTANEOUS LUPUS ERYTHEMATOSUS

(75) Inventors: Hans-Joachim Anders, Munich (DE); Dean Jason Naylor, Eight Mile Plains (AU); Richard James Brown, St. Lucia (AU); Walter Rene Antonius Van Heumen, Bellbowrie (AU)

(73) Assignee: CBIO LIMITED, 8 Mile Plains (AU)

(21) Appl. No.: 12/750,591

(22) Filed: Mar. 30, 2010

(51) Int. Cl. A61K 39/395; A61K 38/16; A61K 33/24; A61P 37/02

(52) U.S. Cl. 424/130.1; 514/21.2; 424/649

(57) ABSTRACT

Disclosed herein, in certain embodiments, are methods and compositions for the prevention and treatment of autoimmune disease. Further disclosed herein, in certain embodiments, are methods for the prevention and treatment of cutaneous lupus erythematosus. Additionally disclosed herein, in certain embodiments, are compositions for the prevention and treatment of cutaneous lupus erythematosus.

### TECHNICAL FIELD

The invention relates generally to the prevention and treatment of autoimmune disease. More specifically, the invention relates to compositions and methods for the prevention and treatment of cutaneous lupus erythematosus.

### BACKGROUND

Cutaneous lupus erythematosus (CLE) (also commonly referred to as “discoid lupus erythematosus” or simply “lupus erythematosus”) comprises a group of diseases affecting the skin and subcutaneous tissue of afflicted individuals. CLE accounts for approximately 10% of all lupus cases. Although a small number of individuals with CLE may eventually develop systemic lupus erythematosus (SLE), CLE is generally confined to the skin in the majority of patients and the systemic pathology associated with SLE does not develop. For example, approximately only 5% of patients with discoid lupus erythematosus go on to develop SLE. Reflecting this, the 10th revision of the World Health Organisation's International Classification of Diseases (ICD-10) classifies CLE under “Diseases of the skin and subcutaneous tissue” (ICD-10 code L93). Code L93 specifically excludes systemic lupus erythematosus (SLE) which is classified as a distinct condition under ICD-10 code M32 (“Diseases of the musculoskeletal system and connective tissue”).

Cutaneous lupus erythematosus usually manifests following UV light exposure (e.g. UV-A and/or UV-B) although other triggers have also been implicated. For example, several drugs may induce CLE, one of the most frequently implicated being hydrochlorothiazide. Calcium channel blockers, tumour necrosis factor antagonists, terbinafine, and angiotensin-converting enzyme inhibitors have also been associated with the induction of CLE. Given its association with UV exposure, CLE often manifests in sun-exposed areas of the skin such as the face, scalp, legs, arms and backs of hands, and can create various kinds of skin lesions. A raised, annular scaly rash is typically formed in CLE. Scarring often develops as the rash heals. In a specific form of CLE known as lupus profundus, inflammation of fat results in the formation of deep nodules on the face and other areas of the body that often persist for several months. This condition may result in lipodystrophy and scar formation due to the destruction of fat cells. CLE may also affect the lips and mouth causing scaling/ulcers and predisposing to squamous cell carcinoma. If hair follicles are involved they may be destroyed (scarring alopecia) causing baldness.

Current treatments for CLE are primarily symptom-relieving drugs with limited effectiveness. Furthermore, many are associated with undesirable side-effects. For example, commonly used treatments such as steroids and corticosteroids are associated with both local (e.g. skin atrophy, acne, loss of pigment, susceptibility to infection) and systemic side effects (e.g. adrenal suppression, osteoporosis, high blood pressure and diabetes). Other commonly used drugs such as retinoids (inflammation, skin sensitivity and irritation), methotrexate (anaemia, neutropenia, hair loss, nausea), clofazamine (skin pigmentation, depression, icthyosis) and ciclosporin (kidney failure, hypertension, elevated blood pressure) are also associated with varying degrees of undesirable side effects.

A general need exists for improved agents and methods to treat cutaneous lupus erythematosus.

### SUMMARY OF THE INVENTION

In a first aspect, the invention provides a method for preventing or treating cutaneous lupus erythematosus in a subject, the method comprising administering a therapeutically effective amount of a chaperonin 10 (Cpn10) polypeptide to the subject.

In one embodiment of the first aspect, the Cpn10 polypeptide comprises an amino acid sequence as set forth in SEQ ID NO: 1 or SEQ ID NO: 2.

In another embodiment of the first aspect, the Cpn10 polypeptide is a variant polypeptide sharing at least 70% sequence identity with a wild-type Cpn10 polypeptide, or a fragment thereof.

In one embodiment of the first aspect, the Cpn10 polypeptide is a variant polypeptide sharing at least 85% sequence identity with a wild-type Cpn10 polypeptide, or a fragment thereof.

In a further embodiment of the first aspect, the variant polypeptide is an N-terminal variant polypeptide comprising an N-terminus extended by at least one additional amino acid residue compared to said wild-type Cpn10 polypeptide.

In another embodiment of the first aspect, the N-terminal variant polypeptide comprises the amino acid sequence set forth in SEQ ID NO: 4, SEQ ID NO: 7, SEQ ID NO: 97 or SEQ ID NO: 106.

In a further embodiment of the first aspect, the N-terminal variant polypeptide comprises the amino acid sequence set forth in any one of SEQ ID NO: 10, SEQ ID NO: 13, SEQ ID NO: 14, SEQ ID NOs: 17-29, SEQ ID NO: 31, SEQ ID NO: 34, SEQ ID NO: 37, SEQ ID NO: 40, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 62, SEQ ID NO: 65, SEQ ID NO: 68, SEQ ID NO: 71, SEQ ID NO: 73, SEQ ID NO: 76, SEQ ID NO: 77, SEQ ID NO:78, SEQ ID NO: 79, SEQ ID NO: 82, SEQ ID NO: 85, SEQ ID NO: 88, SEQ ID NO: 91, or SEQ ID NO: 93.

In one embodiment of the first aspect, the variant polypeptide comprises an increased net positive charge in comparison to a wild-type Cpn10 polypeptide.

In one embodiment of the first aspect, the variant polypeptide comprises an increased net positive charge in comparison to Ala-Cpn10 (SEQ ID NO: 4).

In another embodiment of the first aspect, the variant polypeptide comprises the amino acid sequence set forth in any one SEQ ID NO: 97, SEQ ID NO: 100, SEQ ID NO: 103, or SEQ ID NO: 106.

In one embodiment of the first aspect, the cutaneous lupus erythematosus is selected from the group consisting of chronic cutaneous lupus erythematosus, subacute cutaneous lupus erythematosus, discoid lupus erythematosus, lupus erythematosus NOS, hypertrophic lupus erythematosus, tumid lupus erythematosus, lupus panniculitis, lupus profundus, cutaneous lupus mucinosis, acute cutaneous lupus erythematosus, chronic cutaneous lupus erythematosus, drug-induced lupus erythematosus, and intermittent cutaneous lupus erythematosus.

In an additional embodiment of the first aspect, the method comprises administering at least one additional agent.

In a further embodiment of the first aspect, the additional agent is one or more of topical steroids, corticosteroids, calcineurin inhibitors, antimalarial drugs, retinoids, methotrexate, thalidomide, ciclosporin, dapsone, gold, clofazamine, cyclophosphamide, and immunoglobulin.

In a second aspect, the invention provides use of a chaperonin 10 (Cpn10) polypeptide in the preparation of a medicament for the prevention or treatment of cutaneous lupus erythematosus.

In one embodiment of the second aspect, the Cpn10 polypeptide comprises an amino acid sequence as set forth in SEQ ID NO: 1 or SEQ ID NO: 2.

In one embodiment of the second aspect, the Cpn10 polypeptide is a variant polypeptide sharing at least 70% sequence identity with a wild-type Cpn10 polypeptide, or a fragment thereof.

In another embodiment of the second aspect, the variant polypeptide is an N-terminal variant polypeptide comprising an N-terminus extended by at least one additional amino acid residue compared to said wild-type Cpn10 polypeptide.

In another embodiment of the second aspect, the N-terminal variant polypeptide comprises an amino acid sequence as set forth in SEQ ID NO: 4, SEQ ID NO: 7, SEQ ID NO: 10, SEQ ID NO: 13, SEQ ID NO: 14, SEQ ID NOs: 17-29, SEQ ID NO: 31, SEQ ID NO: 34, SEQ ID NO: 37, SEQ ID NO: 40, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 62, SEQ ID NO: 65, SEQ ID NO: 68, SEQ ID NO: 71, SEQ ID NO: 73, SEQ ID NO: 76, SEQ ID NO: 77, SEQ ID NO:78, SEQ ID NO: 79, SEQ ID NO: 82, SEQ ID NO: 85, SEQ ID NO: 88, SEQ ID NO: 91, SEQ ID NO: 93, SEQ ID NO: 97, or SEQ ID NO: 106.

In one embodiment of the second aspect, the variant polypeptide comprises an increased net positive charge in comparison to a wild-type Cpn10 polypeptide.

In one embodiment of the second aspect, the variant polypeptide comprises an increased net positive charge in comparison to Ala-Cpn10 (SEQ ID NO: 4).

In one embodiment of the second aspect, the variant polypeptide comprises the amino acid sequence set forth in any one SEQ ID NO: 97, SEQ ID NO: 100, SEQ ID NO: 103, or SEQ ID NO: 106.

In another embodiment of the second aspect, the medicament comprises at least one additional agent.

In one embodiment of the second aspect, the additional agent is selected from the group consisting of topical steroids, corticosteroids, calcineurin inhibitors, antimalarial drugs, retinoids, methotrexate, thalidomide, ciclosporin, dapsone, gold, clofazamine, cyclophosphamide, and immunoglobulin.

In another embodiment of the second aspect, the cutaneous lupus erythematosus is selected from the group consisting of chronic cutaneous lupus erythematosus, subacute cutaneous lupus erythematosus, discoid lupus erythematosus, lupus erythematosus NOS, hypertrophic lupus erythematosus, tumid lupus erythematosus, lupus panniculitis, lupus profundus, cutaneous lupus mucinosis, acute cutaneous lupus erythematosus, chronic cutaneous lupus erythematosus, drug-induced lupus erythematosus, and intermittent cutaneous lupus erythematosus.

In a third aspect, the invention provides a composition comprising a chaperonin 10 (Cpn10) polypeptide and at least one additional agent for the treatment of cutaneous lupus erythematosus.

In one embodiment of the third aspect, the additional agent is selected from the group consisting of topical steroids, corticosteroids, calcineurin inhibitors, antimalarial drugs, retinoids, methotrexate, thalidomide, ciclosporin, dapsone, gold, clofazamine, cyclophosphamide, and immunoglobulin.

In one embodiment of the third aspect, the Cpn10 polypeptide comprises an amino acid sequence as set forth in SEQ ID NO: 1 or SEQ ID NO: 2.

In one embodiment of the third aspect, the Cpn10 polypeptide is a variant polypeptide sharing at least 70% sequence identity with a wild-type Cpn10 polypeptide, or a fragment thereof.

In another embodiment of the third aspect, the variant polypeptide is an N-terminal variant polypeptide comprising an N-terminus extended by at least one additional amino acid residue compared to said wild-type Cpn10 polypeptide.

In one embodiment of the third aspect, the variant polypeptide comprises an increased net positive charge in comparison to a wild-type Cpn10 polypeptide.

In one embodiment of the third aspect, the variant polypeptide comprises an increased net positive charge in comparison to Ala-Cpn10 (SEQ ID NO: 4).

In another embodiment of the third aspect, the variant polypeptide comprises an amino acid sequence as set forth in any one of SEQ ID NO: 4, SEQ ID NO: 7, SEQ ID NO: 10, SEQ ID NO: 13, SEQ ID NO: 14, SEQ ID NOs: 17-29, SEQ ID NO: 31, SEQ ID NO: 34, SEQ ID NO: 37, SEQ ID NO: 40, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 62, SEQ ID NO: 65, SEQ ID NO: 68, SEQ ID NO: 71, SEQ ID NO: 73, SEQ ID NO: 76, SEQ ID NO: 77, SEQ ID NO:78, SEQ ID NO: 79, SEQ ID NO: 82, SEQ ID NO: 85, SEQ ID NO: 88, SEQ ID NO: 91, SEQ ID NO: 93, SEQ ID NO: 97, SEQ ID NO: 100, SEQ ID NO: 103, or SEQ ID NO: 106.

In a fourth aspect, the invention provides a chaperonin 10 (Cpn10) polypeptide for the prevention or treatment of cutaneous lupus erythematosus.

### DEFINITIONS

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”. For example, a composition “comprising” a polypeptide may consist exclusively of that polypeptide or may include one or more additional components (e.g. other different polypeptides).

As used herein, the terms “cutaneous lupus erythematosus” and “CLE” specifically exclude systemic lupus erythematosus (SLE).

The term “therapeutically effective amount” as used herein, includes within its meaning a non-toxic but sufficient amount of a compound or composition for use in the invention to provide the desired therapeutic effect. The exact amount required will vary from subject to subject depending on factors such as the species being treated, the age and general condition of the subject, the severity of the condition being treated, the particular agent being administered, the mode of administration and so forth. Thus, it is not possible to specify an exact “effective amount”. However, for any given case, an appropriate “effective amount” may be determined by one of ordinary skill in the art using only routine experimentation.

As used herein, an “agent” includes within its scope any natural or manufactured element or compound. Accordingly, the term includes, but is not limited to any chemical elements and chemical compounds, nucleic acids, amino acids, polypeptides, proteins, antibodies and fragments of antibodies, and other substances that may be appropriate in the context of the invention.

As used herein, the term “administering” and variations of that term including “administer” and “administration”, includes contacting, applying, delivering or providing a compound or composition of the invention to an organism by any appropriate means.

As used herein, the term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

As used herein, the term “polypeptide” or “peptide” means a polymer made up of amino acids linked together by peptide bonds. The terms “polypeptide” and “peptide” are used interchangeably herein, although for the purposes of the present invention a “polypeptide” may constitute a portion of a full length protein. A “peptide” or “polypeptide” of the invention encompasses variants and fragments thereof.

As used herein, the term “polynucleotide” refers to a single- or double-stranded polymer of deoxyribonucleotide, ribonucleotide bases or known analogues or natural nucleotides, or mixtures thereof. A “polynucleotide” of the invention encompasses variants and fragments thereof.

As used herein the term “treatment” refers to any and all uses which remedy a disease state or symptoms, or otherwise prevent, hinder, retard, or reverse the progression of disease or other undesirable symptoms in any way whatsoever.

As used herein, the term “subject” includes humans and individuals of any mammalian species of social, economic or research importance including but not limited to members of the genus ovine, bovine, equine, porcine, feline, canine, primates, and rodents. A subject in accordance with the invention may be a mammal. The mammal may be a human.

Unless the context requires otherwise or specifically stated to the contrary, integers, steps, or elements of the invention recited herein as singular integers, steps or elements encompass both singular and plural forms of the recited integers, steps or elements.

Any description of prior art documents herein, or statements herein derived from or based on those documents, is not an admission that the documents or derived statements are part of the common general knowledge of the relevant art.

For the purposes of description all documents referred to herein are incorporated by reference in their entirety unless otherwise stated.

### BRIEF DESCRIPTION OF THE DRAWINGS

FIG. 1 provides a graph showing survival rate in MRL_{lpr/lpr} mice treated with Ala-Cpn10-Y75K (▴ symbol), Gly-Cpn10 (♦ symbol) or formulation buffer control (▪ symbol). x-axis: percentage survival; y-axis: age (weeks); * P<0.05.

FIG. 2 is a graph showing spleen weight as a proportion of body weight in MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: percentage spleen weight/100 g body weight.

FIG. 3 is a graph showing lymph node weight as a proportion of body weight in MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: percentage lymph node weight/100 g body weight.

FIG. 4 is a representative fluorescence-activated cell sorting (FACS) dotplot showing the proportion of CD3⁺CD4⁻CD8⁻ cells in splenocytes from an MRL^{lpr/lpr} mouse treated with formulation buffer control. x-axis: CD3⁺CD4⁺; y-axis: CD3⁺CD8⁺.

FIG. 5 is a graph showing the proportion of CD3⁺CD4⁻CD8⁻ cells as a percentage of total splenocytes from MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: percentage of CD3⁺CD4⁻CD8⁻ in total cells.

FIG. 6 is a representative fluorescence-activated cell sorting (FACS) dotplot showing the proportion of CD4⁺CD25⁺regulatory T cells in splenocytes from an MRL mouse treated with formulation buffer control. x-axis: CD3⁺CD4⁺; y-axis: CD3^{+CD}25⁺.

FIG. 7 is a graph showing the proportion of CD4⁺CD25^{+ regulatory T cells in total T cells from MRL}^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: percentage of CD4⁺CD25⁺ T cells in total T cells.

FIG. 8 is a graph showing total IgG in serum in MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: total IgG mg/ml.

FIG. 9 is a graph showing total ds-DNA IgG in serum in MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: total ds-DNA IgG mg/ml.

FIG. 10 is a graph showing levels of IFNγ in plasma from MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: plasma IFNγ.

FIG. 11 is a graph showing levels of TNFα in plasma from MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: plasma TNFα.

FIG. 12 is a graph showing levels of IL-10 in plasma from MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: plasma IL-10.

FIG. 13 is a graph showing the albumin/creatinine ratio in urine of MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: albumin/creatinine ratio. * P<0.05.

FIG. 14 is a graph showing creatinine levels in plasma of MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: creatinine levels in plasma (mg/dl). * P<0.05.

FIG. 15 is a graph showing the renal activity index of MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: activity index. * P=0.05; ** P<0.05.

FIG. 16 is a graph showing the renal chronicity index of MRL^{lpr/lpr} mice treated with formulation buffer control (1), Gly-Cpn10 100 μg (2), Gly-Cpn10 200 μg (3), Ala-Cpn10-Y75K 100 μg (4) or Ala-Cpn10-Y75K 200 μg (5). x-axis: formulation/compound administered; y-axis: activity index. * P=0.05.

FIGS. 17A and 17B are photographs of MRL^{lpr/lpr} mice treated with formulation buffer control (*) or Gly-Cpn10/Ala-Cpn10-Y75K 100 μg (**).

FIGS. 18A-18C provide images of histological sections from the skin of MRL^{lpr/lpr} mice treated with formulation buffer control (A), Ala-Cpn10-Y75K (B), or Gly-Cpn10 (C). Skin sections were treated with Periodic Acid Schiff (PAS) stain.

FIG. 18D is a graph showing skin histology scores based on skin sections taken from MRL^{lpr/lpr} mice treated with formulation buffer control (1), Gly-Cpn10 100 μg (2), Gly-Cpn10 200 μg (3), Ala-Cpn10-Y75K 100 μg (4), or Ala-Cpn10-Y75K 200 μg (5). x-axis: formulation/compound administered; y-axis: skin histology score. *** P<0.005.

### DETAILED DESCRIPTION

The present invention relates to the prevention and/or treatment of cutaneous lupus erythematosus (CLE). As demonstrated in the experimental data provided herein, chaperonin 10 (Cpn10), also known as heat shock protein 10 kDa (Hsp10), is an effective agent for preventing and/or treating CLE. Accordingly, the invention provides pharmaceutical compositions comprising Cpn10 for administration to individuals suffering from CLE, or for administration to individuals suspected or identified as having a predisposition to developing CLE. The invention also provides methods for the prevention and/or treatment of CLE.

### Chaperonin 10

The present invention provides Cpn10 polypeptides useful for the prevention and/or treatment of CLE, and polynucleotides encoding the same.

Typically, a polypeptide of the invention is an isolated polypeptide. Similarly, a polynucleotide of the invention is typically an isolated polynucleotide. It will be understood that the term “isolated” in this context means that the polypeptide or polynucleotide has been removed from or is not associated with some or all of the other components with which it would be found in its natural state. For example, an “isolated” polypeptide may be removed from other amino acid residues within a larger polypeptide sequence, or may be removed from natural components such as unrelated proteins. An “isolated” polynucleotide may be removed from other nucleic acid residues within a larger polynucleotide sequence, or may be removed from natural components such as unrelated nucleic acids. For the sake of clarity, an “isolated” polypeptide or polynucleotide also includes a polypeptide or polynucleotide which has not been taken from nature but rather has been prepared de novo, such as chemically synthesised and/or prepared by recombinant methods. As described herein an isolated Cpn10 polypeptide of the invention may be included as a component part of a longer polypeptide or fusion protein.

A polypeptide or polynucleotide of the invention may be derived from, or share sequence homology with, a Cpn10 polypeptide or polynucleotide derived from any organism. Non-limiting examples of suitable organisms include plants (e.g. Arabidopsis thaliana), bacteria (e.g. Mycobacterium tuberculosis, Escherichia coli), yeast (e.g. Saccharomyces cerevisiae), nematodes (e.g. Caenorhabditis elegans), and animals such as mammals, frogs (e.g. Xenopus tropicalis), fruit flies (e.g. Drosophila melanogaster), chicken, fish (e.g. Danio terio) and other marine animals (e.g. sea squirts such as Ciona savignyi).

Preferably, polypeptides and polynucleotides of the invention are derived from, or share sequence homology with, a eukaryotic Cpn10 polypeptide or polynucleotide and more preferably a mammalian Cpn10 polypeptide or polynucleotide. Preferably, polypeptides and polynucleotides of the invention are derived from, or share sequence homology with, a human Cpn10 polypeptide or Cpn10 polynucleotide.

### Cpn10 Polypeptides

Cpn10 polypeptides of the invention (also referred to hereinafter as “polypeptides of the invention”) may be isolated from an organism comprising the polypeptides. It will be understood that isolating polypeptides from an organism in this context includes isolating polypeptides from tissues and or cells derived from the organism. The cells and tissues may be cultured cells and tissues. The isolated polypeptides may be purified during and/or after the isolation process. Methods for the isolation and purification of proteins from organisms (including cells and tissues thereof) are known in the art and described in standard texts (see, for example, Coligan et al., (2000-2010), “Current Protocols in Protein Science”, (Chapter 6), John Wiley and Sons, Inc; Sambrook et al., (1989), “Molecular Cloning: A Laboratory Manual”, Cold Spring Harbor Laboratory Press, Plainview, N.Y.; Ausubel et al., (Eds) (2000-2010), “Current Protocols in Molecular Biology”, John Wiley and Sons, Inc; and, Coico et al (Eds) (2000-2010), “Current Protocols in Microbiology”, John Wiley and Sons, Inc).

Additionally or alternatively, Cpn10 polypeptides of the invention may be produced by other means known in the art. For example, the polypeptides may be produced by conventional methods used in peptide chemistry synthesis such as solid phase peptide synthesis (see, for example, Steward et al., (1963), in “Solid Phase Peptide Synthesis”, H. Freeman Co., San Francisco; and Meienhofer, (1973), in “Hormonal Proteins and Peptides”, volume 2, 46), or by classical solution synthesis (see, for example, Schroder et al., (1965), in “The Peptides”, volume 1, 72-75, Academic Press, New York). A polypeptide of the invention may be produced using recombinant polypeptide production techniques known in the art and described in detail, for example, in standard texts such as Coligan et al., (Eds) (2007), “Current Protocols in Protein Science”, (Chapter 5), John Wiley and Sons, Inc.; and Pharmacia Biotech., (1994), “The Recombinant Protein Handbook”, Pharmacia Biotech. Commonly used expression systems that may be used for the production of polypeptides of the invention include, for example, bacterial (e.g. E. coli), yeast (e.g. Saccharomyces cerevisiae, Aspergillus, Pichia pastorisis), viral (e.g. baculovirus and vaccinia), cellular (e.g. mammalian and insect) and cell-free systems. Suitable cell-free systems that may be used include, but are not limited to, eukaryotic rabbit reticuloctye, wheat germ extract systems, and the prokaryotic E. coli cell-free system (see, for example, Madin et al., Proc. Natl. Acad. Sci. U.S.A. 97: 559-564 (2000), Pelham and Jackson, Eur. J. Biochem., 67: 247-256 (1976); Roberts and Paterson, Proc. Natl. Acad. Sci., 70: 2330-2334 (1973); Zubay, Ann. Rev. Genet., 7: 267 (1973); Gold and Schweiger, Meth. Enzymol., 20: 537 (1971); Lesley et al., J. Biol. Chem., 266(4): 2632-2638 (1991); Baranov et al., Gene, 84: 463-466 (1989); and Kudlicki et al., Analyt. Biochem., 206: 389-393 (1992)).

It will also be understood that polypeptides of the invention may be produced, by digestion of a protein or larger polypeptide with one or more proteinases (e.g. endoLys-C, endoArg-C, endoGlu-C and Staphylococcus V8-protease). Digested peptide fragments can be purified using known techniques such as, for example, high performance liquid chromatographic (HPLC).

A polypeptide of the invention may be derived from, or share sequence homology with, a naturally-occurring form of Cpn10 (i.e. a “wild-type” Cpn10 polypeptide) derived from any organism.

In certain embodiments, polypeptides of the invention may be wild-type human Cpn10 polypeptides. The skilled addressee will recognise that although the human wild-type Cpn10 polypeptide is initially formed with an N-terminal methionine residue, it is generally cleaved under normal biological conditions due to the presence of the second alanine residue (i.e. the “N-end rule”). Accordingly, a human wild-type Cpn10 polypeptide as contemplated herein may or may not commence with a methionine residue.

In certain embodiments, Cpn10 polypeptides of the invention comprise the amino acid sequence set forth in SEQ ID NO: 1 (Met-Cpn10) or (SEQ ID NO: 2) (X-Cpn10). In other embodiments, Cpn10 polypeptides of the invention comprise an amino acid sequence that is a variant or a fragment the amino acid sequence set forth in SEQ ID NO: 1 or SEQ ID NO: 2.

A Cpn10 polypeptide of the invention may be an “N-terminal variant”. As contemplated herein, an “N-terminal variant” differs from a wild-type Cpn10 polypeptide sequence by the substitution, deletion and/or addition of one or more amino acid residue(s) in or to the N-terminal domain. It will be understood that the “N-terminal domain” of Cpn10 encompasses all amino acid residues in the polypeptide sequence prior to those residues which form the first β-strand of the β-barrel in the Cpn10 monomer. Accordingly, the “N-terminal domain” of the human wild-type Cpn10 sequence may be defined by residues 1-8 of the amino acid sequence set forth in SEQ ID NO: 1, or by residues 1-7 of the amino acid sequence set forth in SEQ ID NO: 2.

A Cpn10 polypeptide of the invention that is an “N-terminal variant” may thus be generated by adding, substituting or deleting any amino acid residue(s) in or to the N-terminal domain of a wild-type Cpn10 polypeptide sequence, or a variant or a fragment of that polypeptide sequence (i.e. a variant or fragment thereof). Any number/type of amino acid(s) may be added, substituted or deleted in the N-terminal domain.

In certain embodiments, the N-terminal variant comprises an amino acid sequence as set forth in SEQ ID NO: 2 (or a variant or fragment thereof) and further comprises a sequence of one or more additional residue(s) appended to the N-terminus alanine residue of SEQ ID NO: 2. The appended sequence may comprise any number of additional amino acid residue(s) including, for example, one, two, three or four additional residues. No limitation exists as to the specific identity of the additional amino acid residue(s) which may be positively charged, negatively charged, or neutral residues. In certain embodiments, the first N-terminal residue of the N-terminal variant is a methionine residue.

Non-limiting examples of N-terminal variants include those disclosed in Australian provisional patent application number AU2009904956 entitled “Chaperonin 10 variants”, the contents of which are hereby incorporated by reference in their entirety.

Preferred N-terminal variants include, but are not limited to, variants comprising the amino acid sequence set forth in SEQ ID NO: 4 (Ala-Cpn10), SEQ ID NO: 7 (Gly-Cpn10), SEQ ID NO: 97 (Ala-Cpn10-Y75K), SEQ ID NO: 106 (MA-Cpn10-Y75K), or variants or fragments thereof.

Other additional examples of N-terminal variants include, but are not limited to, variants comprising the amino acid sequence set forth in SEQ ID NO: 10 (Pro-Cpn10), SEQ ID NO: 13 (His-Cpn10), SEQ ID NO: 14 (Ser-Cpn10), SEQ ID NO: 17 (Val-Cpn10), SEQ ID NO: 18 (Leu-Cpn10), SEQ ID NO: 19 (Ile-Cpn10), SEQ ID NO: 20 (Phe-Cpn10), SEQ ID NO: 21 (Tyr-Cpn10), SEQ ID NO: 22 (Trp-Cpn10), SEQ ID NO: 23 (Cys-Cpn10), SEQ ID NO: 24 (Thr-Cpn10), SEQ ID NO: 25 (Asp-Cpn10), SEQ ID NO: 26 (Asn-Cpn10), SEQ ID NO: 27 (Glu-Cpn10), and SEQ ID NO: 28 (Gln-Cpn10), or variants or fragments thereof.

Further additional examples of N-terminal variants include, but are not limited to, variants comprising the amino acid sequence set forth in any one of SEQ ID NO: 29, SEQ ID NO: 31, SEQ ID NO: 34, SEQ ID NO: 37, SEQ ID NO: 40, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 62, SEQ ID NO: 65, SEQ ID NO: 68, SEQ ID NO: 71, SEQ ID NO: 73, SEQ ID NO: 76, SEQ ID NO: 77, SEQ ID NO:78, SEQ ID NO: 79, SEQ ID NO: 82, SEQ ID NO: 85, SEQ ID NO: 88, SEQ ID NO: 91 or SEQ ID NO: 93, or variants or fragments thereof.

A polypeptide of the invention may differ from a wild-type Cpn10 polypeptide sequence by the substitution, deletion and/or insertion of one or more amino acid residue(s) in or to the N-terminus. Additionally or alternatively, a polypeptide of the invention may differ from a wild-type Cpn10 polypeptide sequence by the substitution, deletion and/or insertion of one or more amino acid residue(s) in or to the roof loop (e.g. residues 53-63 of SEQ ID NO: 1; e.g. residues 52-62 of SEQ ID NO: 2), mobile loop (e.g. residues 22-39 of SEQ ID NO: 1; e.g. residues 21-38 of SEQ ID NO: 2), β-strand(s) (i.e. β-strands 1, 2, 3, 4 and/or 5; e.g. residues 9-21, 42-52, 64-74, 78-81, and 86-101 respectively of SEQ ID NO: 1; e.g. residues 8-20, 41-51, 63-73, 77-80, and 85-100 respectively of SEQ ID NO: 2), and/or connective loop(s) (i.e. connective loop 1, 2, and/or 3; e.g. residues 41-42, 75-77, and 82-85 respectively of SEQ ID NO: 1; e.g. residues 40-41, 74-76, and 81-84 respectively of SEQ ID NO: 2) of a wild-type Cpn10 polypeptide sequence, or a variant or fragment thereof. Any number/type of amino acid(s) may be added, substituted or deleted in the aforementioned regions of the wild-type Cpn10 polypeptide.

It will be understood that apart from modifications in one or more of the N-terminus, roof loop, mobile loop, β-strand(s), and/or connective loop(s), a Cpn10 polypeptide of the invention may otherwise be identical or substantially identical to a wild-type Cpn10 polypeptide, or a fragment or variant thereof. Preferably, the wild-type Cpn10 polypeptide is derived from a mammal. For example, the wild-type Cpn10 polypeptide may be a primate, ovine, bovine, equine, porcine, feline, canine, or murine Cpn10 polypeptide. More preferably, the wild-type Cpn10 polypeptide is a mammalian wild-type Cpn10 polypeptide and more preferably a human wild-type Cpn10 polypeptide.

In certain embodiments, a Cpn10 polypeptide of the invention possesses an increased net-positive charge (at physiological pH) in comparison to a wild-type Cpn10 polypeptide and/or in comparison to Ala-Cpn10. In general, the positive charge of a wild-type Cpn10 polypeptide (or Ala-Cpn10) may be increased by the insertion of one or more positively-charged amino acid residue(s), the deletion of one or more negatively-charged amino acid residues, the substitution of one or more negatively charged amino acid residue(s) with a neutral or positively-charged amino acid residue, and/or the substitution of one or more neutral amino acid residue(s) with a positively-charged amino acid residue. Preferably, the substituted, deleted and/or inserted residue(s) are surface/solution-exposed residues in the tertiary structure of the Cpn10 polypeptide.

The net charge of a polypeptide at a given pH may be calculated on the basis of the Henderson-Hasselbalch equation (Hasselbalch, (1917), “Die Berechnung der Wasserstoffzahl des Blutes aus der freien und gebundenen Kohlensäure desselben, und die Sauerstoffbindung des Blutes als Funktion der Wasserstoffzahl”, Biochemische Zeitschrift 78: 112-144) and known pKa values of ionisable amino acid side chains and the ionisable carboxyl and amino termini of a polypeptide. The pKa values of ionisable groups within a polypeptide are well known in the art can be found in standard texts. Depending on the set of pKa values used, the calculated net charge of a protein may vary slightly, but this does not change the conclusions obtained in this study. The pKa values utilised to calculate the net charge of each polypeptide listed in Table 1 below are as follows: N-terminus 8.0, C-terminus 3.1, Lys 10.0, Arg 12.0, His 6.5, Glu 4.4, Asp 4.4, Tyr 10.0 and Cys 8.5 (see Stryer, (1988), “Biochemistry”, 3^{rd} Edition, New York, W.H. Freeman, ISBN 0716719207).

TABLE 1
net charge of Cpn10 and exemplary Cpn10 variants at pH 7.3 and pH 7.4.

| | Net charge | |
|---|---|---|
| Protein | at pH 7.3 | Net charge at pH 7.4 |
| M-Cpn10 (SEQ ID NO: 1) | 1.8 | 1.8 |
| X-Cpn10 (SEQ ID NO: 2) | 1.8 | 1.8 |
| Ala-Cpn10 (SEQ ID NO: 4) | 1.8 | 1.8 |
| Gly-Cpn10 (SEQ ID NO: 7) | 1.8 | 1.8 |
| Ala-Cpn10-Y75K (SEQ ID NO: 97) | 2.8 | 2.8 |
| X-Cpn10-Y75K (SEQ ID NO: 100) | 2.8 | 2.8 |
| M-Cpn10-Y75K (SEQ ID NO: 103) | 2.8 | 2.8 |
| MA-Cpn10-Y75K (SEQ ID NO: 106) | 2.8 | 2.8 |

In certain embodiments, a Cpn10 polypeptide of the invention possessing an increased net-positive charge (at physiological pH) in comparison to a wild-type Cpn10 polypeptide and/or in comparison to Ala-Cpn10 may comprise the amino acid sequence set forth in SEQ ID NO: 97 (Ala-Cpn10-Y75K), SEQ ID NO: 100 (X-Cpn10-Y75K), SEQ ID NO: 103 (M-Cpn10-Y75K), SEQ ID NO: 106 (MA-Cpn10-Y75K), or a variant or fragment thereof.

Non-limiting examples of Cpn10 polypeptides included within the scope of the invention include those disclosed in PCT publication number WO/2007/025343 and U.S. application Ser. No. 11/991,279 each entitled “Modified Chaperonin 10”, the contents of which are hereby incorporated by reference in their entirety.

Non-limiting examples of Cpn10 polypeptides included within the scope of the invention also include those disclosed in PCT publication number WO/2009/124353 entitled “Modified Chaperonin 10 and PRR signalling”, the contents of which are hereby incorporated by reference in their entirety.

Polypeptides of the invention may be modified with an acetyl group, lipids, carbohydrates and/or phosphate groups, for example, to improve immunogenicity, stability and/or solubility. Capping of polypeptide termini may be used to enhance stability against cellular proteases.

It will be understood that amino acid residues of polypeptides of the invention include any and all of their isomers (e.g. D-form, L-form and DL-form) and isoforms.

### Polynucleotides

Also included within the scope of the invention are polynucleotides encoding polypeptides of the invention, and polynucleotides encoding variants and fragments of polypeptides of the invention (collectively referred to hereinafter as “polynucleotide(s) of the invention”).

Polynucleotides of the invention may be isolated from an organism comprising the polynucleotides. It will be understood that isolating polynucleotides from an organism in this context includes isolating polynucleotides from tissues and or cells derived from the organism. The cells and tissues may be cultured cells and tissues. The isolated polynucleotides may be purified during and/or after the isolation process. Methods for the isolation and purification of nucleic acids from organisms (including cells and tissues thereof) are known in the art and described in standard texts (see, for example, Sambrook et al., (1989), “Molecular Cloning: A Laboratory Manual”, Cold Spring Harbor Laboratory Press, Plainview, N.Y.; Ausubel et al., (Eds) (2000-2010), “Current Protocols in Molecular Biology”, John Wiley and Sons, Inc.)

Polynucleotides of the invention may be manufactured using standard techniques known in the art such as those described, for example, in Sambrook et al., (1989) “Molecular Cloning: A Laboratory Manual”, (2nd ed., Cold Spring Harbor Laboratory Press, Plainview, N.Y.; Itakura K. et al., (1984), “Synthesis and use of synthetic oligonucleotides”, Annu. Rev. Biochem. 53: 323; Innis et al., (Eds), (1990), “PCR Protocols: A Guide to Methods and Applications”, Academic Press, New York; Innis and Gelfand, (Eds), (1995), “PCR Strategies”, Academic Press, New York; and Innis and Gelfand, (Eds), (1999), “PCR Methods Manual”, Academic Press, New York.

Polynucleotides of the invention can be manufactured, for example, by chemical synthesis techniques including the phosphodiester and phosphotriester methods (see, for example, Narang et al., (1979), “Improved phosphotriester method for the synthesis of gene fragments”, Meth. Enzymol. 68: 90; Brown et al., (1979), “Chemical Synthesis and Cloning of a Tyrosine tRNA Gene”, Meth. Enzymol. 68: 109-151; and U.S. Pat. No. 4,356,270) or the diethylphosphoramidite method (see Beaucage and Caruthers, (1981), “Deoxynucleotide phosphoramidite”, Tetrahedron Letters, 22: 1859-1862). A method for synthesising oligonucleotides on a modified solid support is described in U.S. Pat. No. 4,458,066.

In certain embodiments, polynucleotides of the invention comprise the nucleic acid sequence forth in SEQ ID NO: 3, SEQ ID NO: 5, SEQ ID NO: 6, SEQ ID NO: 8, SEQ ID NO: 9, SEQ ID NO: 11, SEQ ID NO: 12, SEQ ID NO: 15, SEQ ID NO: 16, SEQ ID NO: 30, SEQ ID NO: 32, SEQ ID NO: 33, SEQ ID NO: 35, SEQ ID NO: 36, SEQ ID NO: 38, SEQ ID NO: 39, SEQ ID NO: 41, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 63, SEQ ID NO: 64, SEQ ID NO: 66, SEQ ID NO: 67, SEQ ID NO: 69, SEQ ID NO: 70, SEQ ID NO: 72, SEQ ID NO: 74, SEQ ID NO: 75, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, or a variant or fragment thereof.

Preferably, a polynucleotide sequence of the invention comprises a nucleic acid sequence as set forth in SEQ ID NO: 3, SEQ ID NO: 5, SEQ ID NO: 6, SEQ ID NO: 8, SEQ ID NO: 9, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, or a variant or fragment thereof.

As will be recognized by the skilled artisan, polynucleotides of the invention may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the invention, and a polynucleotide may, but need not, be linked to other molecules and/or support materials. Polynucleotides may comprise a native sequence (i.e. an endogenous sequence that encodes protein or a portion thereof) or may comprise a variant, or a biological or antigenic functional equivalent of such a sequence. Polynucleotide variants may contain one or more substitutions, additions, deletions and/or insertions, as further described below (see section entitled “Variants and fragments”), preferably such that the immunogenicity of the encoded polypeptide is not diminished.

RNA may be derived from RNA polymerase-catalyzed transcription of a DNA sequence. The RNA may be a primary transcript derived from transcription of a corresponding DNA sequence. RNA may also undergo post-transcriptional processing. For example, a primary RNA transcript may undergo post-transcriptional processing to form a mature RNA. Messenger RNA (mRNA) refers to RNA derived from a corresponding open reading frame that may be translated into a protein by the cell. cDNA refers to a double-stranded DNA that is complementary to and derived from mRNA. Sense RNA refers to an RNA transcript that includes the mRNA and so can be translated into protein by the cell. Antisense RNA refers to an RNA transcript that is complementary to all or part of a target primary transcript of mRNA, and may be used to block the expression of a target gene.

The skilled addressee will recognise that RNA and cDNA sequences may be derived using the genetic code. Accordingly, an RNA sequence may be derived from a given DNA sequence by generating a sequence that is complementary to the particular DNA sequence. A complementary DNA (cDNA) sequence may be derived from a DNA sequence by deriving an RNA sequence from the DNA sequence as above, then converting the RNA sequence into a cDNA sequence.

Polynucleotides of the invention may be cloned into a vector. The vector may comprise, for example, a DNA, RNA or complementary DNA (cDNA) sequence. The vector may be a plasmid vector, a viral vector, or any other suitable vehicle adapted for the insertion of foreign sequences, their introduction into cells and the expression of the introduced sequences. Typically the vector is an expression vector and may include expression control and processing sequences such as a promoter, an enhancer, ribosome binding sites, polyadenylation signals and transcription termination sequences.

The invention also contemplates host cells transformed by such vectors. For example, polynucleotides of the invention may be cloned into a vector which is transformed into a bacterial host cell such as, for example, E. coli. Methods for the construction of vectors and their transformation into host cells are generally known in the art, and described in, for example, Sambrook et al., (1989), “Molecular Cloning: A Laboratory Manual”, Cold Spring Harbor Laboratory Press, Plainview, N.Y.; and, Ausubel et al., (Eds) (2007), “Current Protocols in Molecular Biology”, John Wiley and Sons, Inc.

### Variants and Fragments

It will be understood that polypeptides of the invention encompass variants of those polypeptides. Similarly, it will be understood that polynucleotides of the invention encompass variants of those polynucleotides.

The term “variant” as used herein refers to a substantially similar sequence. In general, two sequences are “substantially similar” if the two sequences have a specified percentage of amino acid residues or nucleotides that are the same (percentage of “sequence identity”), over a specified region, or, when not specified, over the entire sequence. Accordingly, a “variant” of a polypeptide of the invention or a polynucleotide of the invention may share at least about 40%, 45%, 50%, 55%, 60%, 65%, 70%, 75%, 80%, 83% 85%, 88%, 90%, 93%, 95%, 96%, 97%, 98% or 99% sequence identity with the reference sequence over the full length of the reference sequence or over a fragment of the reference sequence.

In general, polypeptide variants possess qualitative biological activity in common with the polypeptide from which they are derived. Polynucleotide variants generally encode polypeptides which generally possess qualitative biological activity in common with the polypeptide from which they are derived. Also included within the meaning of the term “variant” are homologues of polynucleotides of the invention or homologues of polypeptides of the invention. A polynucleotide homologue is typically from a different species but sharing substantially the same biological function or activity as the corresponding polynucleotide of the invention. A polypeptide homologue is typically from a different species but sharing substantially the same biological function or activity as the corresponding polypeptide of the invention.

Further, the term “variant” also includes analogues of polypeptides of the invention. A polypeptide “analogue” is a polypeptide which is a derivative of a polypeptide of the invention, which derivative comprises addition, deletion, substitution of one or more amino acids, such that the polypeptide retains substantially the same function. The term “conservative amino acid substitution” refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

The percentage of sequence identity between two sequences may be determined by comparing two optimally aligned sequences over a comparison window. A portion of a sequence (e.g. a polypeptide of the invention or a polynucleotide of the invention) in the comparison window may, for example, comprise deletions or additions (i.e. gaps) in comparison to a reference sequence (e.g. one derived from another species) which does not comprise deletions or additions, in order to align the two sequences optimally, or vice versa. A percentage of sequence identity may then be calculated by determining the number of positions at which identical amino acid residues (or nucleotides) occur in both sequences to yield the number of matched positions, dividing the number of matched positions by the total number of positions in the window of comparison and multiplying the result by 100 to yield the percentage of sequence identity.

In the context of two or more polynucleotide or polypeptide sequences, the percentage of sequence identity refers to the specified percentage of amino acid residues or nucleotides that are the same over a specified region (or, when not specified, over the entire sequence) when compared and aligned for maximum correspondence over a comparison window, or designated region as measured using, for example, one of the following sequence comparison algorithms or by manual alignment and visual inspection.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percentage of sequence identity for the test sequence(s) relative to the reference sequence, based on the program parameters.

Methods of alignment of sequences for comparison are known in the art. Optimal alignment of sequences for determination of sequence identity can be achieved conventionally using known computer programs including, but not limited to, CLUSTAL in the PC/Gene program (available from Intelligenetics, Mountain View, Calif.), the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA).

It will be understood that polypeptides of the invention encompass fragments of those polypeptides. Similarly, it will be understood that polynucleotides of the invention encompass fragments of those polynucleotides.

A “fragment” of a polypeptide of the invention is a polypeptide that encodes a constituent or is a constituent of a polypeptide of the invention or variant thereof. Typically the fragment possesses qualitative biological activity in common with the polypeptide of which it is a constituent. Typically, the polypeptide fragment may be at greater than about 5, 10, 15, 20, 25, 30, 35, 40, 45, 50, 55, 60, 65, 70, 75, 80, 85, 90, 95, 100 or greater than 100 amino acid residues in length.

A “fragment” of a polynucleotide of the invention is a polynucleotide that is a constituent of a polynucleotide of the invention or variant thereof. Fragments of a polynucleotide of the invention do not necessarily need to encode polypeptides which retain biological activity. The fragment may, for example, be useful as a hybridisation probe or PCR primer. Typically, the polynucleotide fragment may be greater than about 5, 10, 15, 20, 25, 30, 35, 40, 45, 50, 60, 70, 80, 90, 100, 125, 150, 175, 200, 225, 250, 275, 300, 325, 350, 375, 425, 450, 475, 500, 550, 600, 650, 700, 750, 800, 850, 900, 950, 1000, 1100, 1200, 1300, 1400, 1500, 1750, 2000, 2250, 2500, 2750, 3000, 3250 or 3400 nucleotides in length.

### Compositions

The present invention provides compositions comprising polypeptides of the invention and/or polynucleotides of the invention. Non-limiting examples of suitable polypeptides and polynucleotides are described above in the section entitled “Chaperonin 10”. It will be understood that compositions of the invention may comprise multiple different polypeptides of the invention and/or multiple different polynucleotides of the invention.

In certain embodiments, compositions of the present invention are pharmaceutical compositions. Pharmaceutical compositions of the present invention may be prepared using methods known to those of ordinary skill in the art. Non-limiting examples of suitable methods are described in Gennaro et al. (Eds), (1990), “Remington's Pharmaceutical Sciences”, Mack Publishing Co., Easton, Pa., USA.

A pharmaceutical composition of the present invention may be administered to a recipient in isolation or in combination with other additional therapeutic agent(s). In embodiments where a pharmaceutical composition is administered with additional therapeutic agent(s), the administration may be simultaneous administration or sequential administration (i.e. pharmaceutical composition administration followed by administration of the agent(s) or vice versa).

Pharmaceutical compositions of the invention may comprise a pharmaceutically acceptable carrier, excipient, diluent and/or adjuvant. “Pharmaceutically acceptable” carriers, excipients, diluents and/or adjuvants as contemplated herein are substances which do not produce adverse reaction(s) when administered to a particular recipient such as a human or non-human animal. Pharmaceutically acceptable carriers, excipients, diluents and adjuvants are generally also compatible with other ingredients of the composition. Non-limiting examples of suitable excipients, diluents, and carriers can be found in Rowe et al. (Eds), (2003), “Handbook of Pharmaceutical Excipients” 4th Edition, The Pharmaceutical Press, London, American Pharmaceutical Association, Washington.

Non-limiting examples of pharmaceutically acceptable carriers, excipients or diluents include demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Compositions of the present invention can be administered to a recipient by standard routes, including, but not limited to, parenteral (e.g. intravenous, intraspinal, subcutaneous or intramuscular), oral, mucosal (e.g. intranasal) or topical routes.

Accordingly, compositions of the present invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

Solid forms of compositions of the present invention for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms of compositions of the present invention for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions comprising compositions of the invention for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

For preparation of compositions as injectable solutions or suspensions, non-toxic parenterally acceptable diluents or carriers may be used such as Ringer's solution, isotonic saline, phosphate buffered saline, ethanol and 1,2 propylene glycol.

Emulsions for oral administration may further comprise one or more emulsifying agents. Suitable emulsifying agents include dispersing agents as exemplified above or natural gums such as guar gum, gum acacia or gum tragacanth.

Topical formulations of the present invention comprise an active ingredient(s) (e.g. polypeptides and/or polynucleotides of the invention) together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. For example, sterilisation may be achieved by filtration followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

Compositions of the present invention may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

Compositions of the present invention may be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq.

Supplementary active ingredients, such as adjuvants or biological response modifiers, can also be incorporated into compositions of the invention.

Preferably, an adjuvant will enhance the immune response induced and/or enhanced by component(s) of a given composition thereby improving protective efficacy. Preferably, the adjuvant will enable the induction of protective immunity utilising a lower dose of other active component(s) (e.g. Cpn10 polypeptides of the invention and/or Cpn10 polynucleotides of the invention).

Any suitable adjuvant may be included in compositions of the present invention. For example, an aluminium-based adjuvant may be utilised. Suitable aluminium-based adjuvants include, but are not limited to, aluminium hydroxide, aluminium phosphate and combinations thereof. Other specific examples of aluminium-based adjuvants that may be utilised are described in European Patent No. 1216053 and U.S. Pat. No. 6,372,223.

Oil in water emulsions may be utilised as adjuvants in compositions of the present invention. Oil in water emulsions are well known in the art. In general, the oil in water emulsion will comprise a metabolisable oil, for example, a fish oil, a vegetable oil, or a synthetic oil. Examples of suitable oil in water emulsions include those described in European Patent No. 0399843, U.S. Pat. No. 7,029,678 and PCT Publication No. WO 2007/006939. The oil in water emulsion may be utilised in combination with other adjuvants and/or immuno stimulants.

Non-limiting examples of other suitable adjuvants include immunostimulants such as granulocyte-macrophage colony-stimulating factor (GM-CSF), monophosphoryl lipid A (MPL), cholera toxin (CT) or its constituent subunit, heat labile enterotoxin (LT) or its constituent subunit, toll-like receptor ligand adjuvants such as lipopolysaccharide (LPS) and derivatives thereof (e.g. monophosphoryl lipid A and 3-Deacylated monophosphoryl lipid A), muramyl dipeptide (MDP) and F protein of Respiratory Syncytial Virus (RSV).

Adjuvants in compositions of the present invention typically include emollients, emulsifiers, thickening agents, preservatives, bactericides and buffering agents. Another type of “self adjuvant” is provided by the conjugation of immunogenic peptides to lipids such as the water soluble lipopeptides Pam3Cys or its dipalmitoyl derivative Pam2Cys. Such adjuvants have the advantage of accompanying and immunogenic component into the antigen presenting cell (such as dendritic cells) and thus producing enhanced antigen presentation and activation of the cell at the same time. These agents act at least partly through toll-like receptor 2 (see, for example, Brown and Jackson, (2005), “Lipid based self adjuvanting vaccines”, Current Drug Delivery, 23:83).

Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In certain embodiments, an adjuvant included in a composition of the present invention may induce an immune response predominantly of the TH1 type. Suitable adjuvants for use in eliciting a predominantly TH1-type response include, for example, a combination of monophosphoryl lipid A, preferably 3-de-O-acylated monophosphoryl lipid A (3D-MPL) together with an aluminium salt. For example, the composition or may be formulated with adjuvant AS04 containing aluminium hydroxide (alum) and 3-O-deacylated monophosphorylated lipid A (MPL) such as described in Thoelen et al. (2001), “A prophylactic hepatitis B vaccine with a novel adjuvant system”, Vaccine, 19: 2400-2403. Other known adjuvants which preferentially induce a TH1 type immune response include CpG containing oligonucleotides. The oligonucleotides are characterised in that the CpG dinucleotide is unmethylated. Such oligonucleotides are known to those of ordinary skill in the field and are described, for example, in PCT Publication No. WO 1996/02555. Immunostimulatory DNA sequences are also described, for example, in Sato et al., (1996), “Immunostimulatory DNA sequences necessary for effective intradermal gene immunization”, Science, 273: 352-354.

Another example of an adjuvant is a saponin, preferably QS21 (Aquila Biopharmaceuticals Inc., Framingham, Mass.), which may be used alone or in combination with other adjuvants. For example, an enhanced adjuvant system may be utilised involving the combination of a monophosphoryl lipid A and saponin derivative, such as the combination of QS21 and 3D-MPL as described in PCT Publication No. WO 1994/00153, or a less reactogenic composition where the QS21 is quenched with cholesterol, as described in PCT publication No. WO 1996/33739. Other alternative formulations comprise an oil-in-water emulsion and tocopherol. An adjuvant formulation involving QS21, 3D-MPL and tocopherol in an oil-in-water emulsion is described in PCT Publication No. WO 1995/17210. An adjuvant included in a composition of the invention may include a formulation involving QS21, 3D-MPL and tocopherol in an oil in water emulsion such as described in PCT publication No. WO 1995/17210. In one embodiment a composition of the invention comprises the adjuvant Montanide ISA720 (M-ISA-720; Seppic, Fairfield, N.J.), an adjuvant based on a natural metabolisable oil.

### Therapeutic Methods

The present invention provides methods for the prevention and/or treatment of cutaneous lupus erythematosus (CLE). The methods comprise administering a polypeptide of the invention and/or polynucleotide of the invention to a subject. The polypeptides and/or polynucleotides may be administered to the subject in the form of a pharmaceutical composition of the invention (see section above entitled “Compositions”).

It will be understood that combinations of different polypeptides, polynucleotides and/or compositions of the invention may be administered to a subject in accordance with the methods described herein.

The subject may be an individual of any mammalian species including, but not limited to, members of the genus ovine, bovine, equine, porcine, feline, canine, primates, and rodents. In one embodiment, the subject is a mammal. Preferably, the mammal is a human suffering from CLE or pre-disposed to developing CLE.

The methods of the invention are suitable for treating and/or preventing any type of CLE. Non-limiting examples of CLE that may be treated and/or prevented using the methods of the invention include chronic cutaneous lupus erythematosus, subacute cutaneous lupus erythematosus, discoid lupus erythematosus, lupus erythematosus NOS, hypertrophic lupus erythematosus, tumid lupus erythematosus (also known as lupus erythematosus tumidus), lupus panniculitis, lupus profundus, cutaneous lupus mucinosis, acute cutaneous lupus erythematosus, chronic cutaneous lupus erythematosus, drug-induced lupus erythematosus, and intermittent cutaneous lupus erythematosus.

In certain embodiments, the methods are used for the treatment or prevention of discoid lupus erythematosus. The discoid lupus erythematosus may be characterised by localised or widespread development of red-coloured scaly patches affecting one or more of the cheeks, nose, ears, lips, inside of mouth, upper back, neck, and/or top surface of hands and may result in postinflammatory pigmentation and/or scarring.

In other embodiments, the methods are used for the treatment or prevention of subacute lupus erythematosus, a non-scarring non-atrophy-producing photosensitive dermatosis Skin rashes that develop during subacute lupus erythematosus may be annular or polycyclic (ring-shaped), and/or be characterised by papulosquamous (scaly bumps), vasculitis (purple spots) and/or nodular lumps.

In other embodiments, the methods are used for the treatment or prevention of lupus erythematosus tumidus. The lupus erythematosus tumidus may be characterised by a photosensitive dermal rash presenting with red, swollen, urticaria-like bumps and patches, some of which may be ring-shaped (annular).

In certain embodiments, the methods are used for the treatment or prevention of lupus profundus (also known as lupus panniculitis), a form of CLE affecting fat underlying skin, particularly on the face. Inflammation of the fat during lupus profundus may cause the formation of firm deep nodules in the skin and result in lipodystrophy.

In certain embodiments, the methods are used for the treatment or prevention of hypertrophic lupus erythematosus, a form of CLE characterised by thickened and warty skin resembling viral warts or skin cancers.

In other embodiments, the methods are used for the treatment or prevention of neonatal lupus erythematosus. The neonatal lupus erythematosus may be characterised by a temporary ring-like or annular rash on a newborn infant. The presence of neonatal lupus erythematosus may predispose the newborn infant to the development of congenital heart block.

In other embodiments, the methods are used for the treatment or prevention of cutaneous lupus mucinosis, a condition characterised by deposits of mucin in the dermis and/or papules (small bumps), plaques (flatter patches) and/or nodules (larger bumps) on, for example, the cheeks, upper chest, upper arms and/or back.

It will be understood that CLE as contemplated herein specifically excludes systemic lupus erythematosus (SLE). Accordingly, a subject treated in accordance with the methods of the invention will generally be a patient who is not suffering from SLE.

CLE may be distinguished from SLE by standard clinical parameters known in the art. For example, SLE is classified as a systemic manifestation of lupus affecting multiple organs and tissues while CLE generally restricted to the skin and subcutaneous tissue (see, for example, 10^{th} revision of the World Health Organisation's International Classification of Diseases (ICD-10): (http://apps.who.int/classifications/apps/icd/icd10online/).

The diagnosis of SLE generally requires an individual to present clinical evidence of multi-organ/system disease (i.e. show abnormalities in several different organs). Typical combinations of symptoms indicative of SLE include concurrent presentation of symptoms such as, for example, renal dysfunction (e.g. persistent proteinuria detectable, for example, by urinalysis), neurological disorders (e.g. convulsions and seizures, psychosis, loss of muscle control and/or strength), polyserositis (e.g. pleuritis, peritonitis, and/or pericarditis), haematological disorders (e.g. reticulocytosis, leukopenia, thrombocytopenia), and/or arthritis affecting peripheral joints (e.g. swelling, effusion or tenderness). These symptoms may be detected using standard techniques known in the art.

Patients suffering from SLE generally present with elevated levels of autoantibodies, and in particular antinuclear autoantibodies and/or double-stranded DNA autoantibodies. Non-limiting examples of such autoantibodies include anti-Ro (SS-A) and anti-La (SS-B) autoantibodies. Methods for the detection of such autoantibodies are known in the art.

Although autoantibodies may be increased in patients suffering from SLE, the same or related autoantibodies may also be increased in patients suffering from CLE.

Accordingly, a specific diagnosis of CLE is generally made on the basis of other factors such as abnormalities in the skin and/or subcutaneous tissue of afflicted individuals in the absence or substantial absence of additional feature(s) indicative of systemic disease (i.e. systemic features associated with SLE). An individual suffering from CLE as contemplated herein will thus exhibit the skin manifestations of lupus in the absence or substantial absence of other symptoms indicative of multi-organ involvement that would lead to a diagnosis of SLE. Accordingly, an individual suffering from CLE may be diagnosed by the presence of skin pathology and the absence of SLE confirmed by verifying the absence of systemic symptoms such as those described in the paragraph above.

In certain embodiments, a diagnosis of CLE may be made on the basis of histological factors. For example, a diagnosis of CLE may be made on the basis of one or more of an infiltrate of inflammatory cells (e.g. lymphocytic) around vessels (e.g. subepidermal, perivascular and/or periappendiceal), vacuolar alteration of the basal cell layer, and/or other changes (e.g. follicular plugging, atrophy, mucin abundance). In general, the histological factors referred to above will present in the absence or substantial absence of systemic feature(s) indicative of SLE.

Without limitation to a particular method of diagnosis, a diagnosis of CLE may be made on the basis of a lupus band test performed on a series of skin biopsies. Methods for performing the lupus band test are known in the art and described, for example in Marks, et al. (2006), “Lookingbill and Marks' Principles of Dermatology”, Elsevier, Inc. The lupus band test may be used to detect IgG and/or complement depositions at the dermal-epidermal junction of the biopsy. A diagnosis of CLE may be made on the basis of a series of skin biopsies taken from a patient suspected of suffering from CLE, wherein the series of biopsies comprises a biopsy taken from an affected region of skin (e.g. a region exhibiting lesions, rashes etc) and a biopsy taken from an unaffected region of skin. A patient suffering from CLE may be diagnosed on the basis of a positive lupus band test on the biopsy taken from the affected region of skin and a negative lupus band test on the biopsy taken from the unaffected region of skin. In contrast, a patient suffering from SLE may be diagnosed on the basis of a positive lupus band test on both the biopsy taken from the affected region of skin and the biopsy taken from the unaffected region of skin.

A polypeptide or polynucleotide of the invention (or a composition comprising the same) used in accordance with the methods described herein may be administered to a subject therapeutically or preventively.

In a therapeutic application, the polypeptide, polynucleotide or composition is administered to a subject already suffering from CLE in an amount sufficient to cure or at least partially arrest the disease and its complications. Typically, in therapeutic applications, the treatment would be for the duration of the disease state or condition.

In a preventative application, the polypeptide, polynucleotide or composition is administered to a subject that is not suffering from CLE at the time of administration. It is contemplated that administration of the polypeptide, polynucleotide or composition to an individual suspected of having a pre-disposition to developing CLE may prevent or assist the prevention of CLE in that individual. For example, an individual may be suspected of having a pre-disposition to developing CLE due to genetic factors such as, for example, human leukocyte antigen (HLA) type (e.g. HLA-B8, HLA-DR3, HLA-DRw52, HLA-DQ1) and/or the prevalence of CLE in related individuals/family members.

In certain embodiments, the polypeptide, polynucleotide or composition is administered to a subject in combination with one or more additional agent(s). The additional agents may include other currently utilised drugs against CLE. For example, the polypeptide, polynucleotide or composition may be administered in combination with one or more of topical steroids, corticosteroids, calcineurin inhibitors, antimalarial drugs (e.g. chloroquine, hydroxychloroquine, and quinacrine), retinoids (e.g. acitretin, isoretinoin), methotrexate, thalidomide, ciclosporin, dapsone, gold, clofazamine, cyclophosphamide, immunoglobulin (e.g. intravenous immunoglobulin) and biological response modifiers.

The efficacy of methods for preventing or treating CLE provided herein may be determined using standard techniques.

For therapeutic applications, such a determination will generally rely on establishing whether CLE is cured or at least partially arrested in the treated subject.

For preventative applications, such a determination will generally rely on establishing whether the subject develops CLE over a relevant time period following treatment.

These factors may be established by clinical examination of the subject for symptoms and manifestations of CLE. Additionally or alternatively, diagnostic assays may be performed to detect the presence of absence of autoantibodies associated with the development of CLE.

### Dosages and Routes of Administration

The therapeutically effective dose level for any particular subject will depend upon a variety of factors including, for example, the particular form of CLE being treated and the severity/degree of progression CLE, the subject's characteristics (e.g. age, body weight, general health, sex and diet of the subject), whether the polypeptide, polynucleotide or composition is used as single agent or as a component of a combination therapy, the time of administration, the route of administration, the rate of sequestration of the administered agent(s), the duration of the treatment, the activity of the administered agent(s) employed, or other related factors known in the art.

Various general considerations that may be considered when determining an appropriate dosage of a polypeptide, polynucleotide or composition of the invention are described, for example, in Gennaro et al. (Eds), (1990), “Remington's Pharmaceutical Sciences”, Mack Publishing Co., Easton, Pa., USA; and Gilman et al., (Eds), (1990), “Goodman And Gilman's: The Pharmacological Bases of Therapeutics”, Pergamon Press.

Further, it will be apparent to one of ordinary skill in the art that the optimal quantity and spacing of individual dosages will be determined by the nature and extent of the CLE being treated, the form, route and site of administration, and the nature of the particular subject being treated.

One skilled in the art would be able, by routine experimentation, to determine an effective, non-toxic amount of a polypeptide, polynucleotide or composition of the invention which would be required to effectively prevent or treat CLE.

For example, an optimal dosage may be derived from administering serially diluted preparations comprising a polypeptide, polynucleotide or composition of the invention in conjunction with a suitable testing procedure. Additionally or alternatively, a matrix comprising various different dosages and dosage frequency can be designed and applied to one or more groups of experimental subjects to determine optimal dosages.

Generally, an effective dosage is expected to be in the range of about 0.0001 mg to about 1000 mg of active agent per kg body weight per 24 hours; typically, about 0.001 mg to about 750 mg of active agent of active agent per kg body weight per 24 hours; about 0.01 mg to about 500 mg of active agent per kg body weight per 24 hours; about 0.1 mg to about 500 mg of active agent per kg body weight per 24 hours; about 0.1 mg to about 250 mg of active agent per kg body weight per 24 hours; or about 1.0 mg to about 250 mg of active agent per kg body weight per 24 hours.

More typically, an effective dose range is expected to be in the range about 1.0 mg to about 200 mg of active agent per kg body weight per 24 hours; about 1.0 mg to about 100 mg of active agent per kg body weight per 24 hours; about 1.0 mg to about 50 mg of active agent per kg body weight per 24 hours; about 1.0 mg to about 25 mg of active agent per kg body weight per 24 hours; about 5.0 mg to about 50 mg of active agent per kg body weight per 24 hours; about 5.0 mg to about 20 mg of active agent per kg body weight per 24 hours; or about 5.0 mg to about 15 mg of active agent per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

In many instances, it will be desirable to have several or multiple administrations of a polypeptide, polynucleotide or composition of the invention (or a composition/vaccine comprising the same). For example, administration may occur 1, 2, 3, 4, 5, 6, 7, 8, 9, 10, or more times. The administrations may be from about one to about twelve week intervals, and in certain embodiments from about one to about four week intervals. Periodic re-administration may be desirable in the case of recurrent exposure to a particular infectious microorganism targeted by a composition of the invention.

It will also be apparent to one of ordinary skill in the art that the optimal course of treatment can be ascertained using conventional course of treatment determination tests.

Where two or more therapeutic entities are administered to a subject “in conjunction”, they may be administered in a single composition at the same time, or in separate compositions at the same time or in separate compositions separated in time.

Administration to a subject of a polypeptide, polynucleotide or composition of the invention in accordance with the methods described herein may be performed by any suitable route including, but not limited to, the parenteral (e.g. intravenous, intradermal, subcutaneous or intramuscular), mucosal (e.g. oral or intranasal) or topical route.

Accordingly, a polypeptide, polynucleotide or composition of the invention may be administered in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

Formulations for intranasal administration may be provided in a freeze-dried powder form, in liquid form as nose drops, spray, or suitable for inhalation, as powder, as cream, or as emulsion.

In certain embodiments, a polypeptide, polynucleotide or composition of the invention is in a form suitable for topical administration (e.g. in the form of an ointment, cream or lotion).

In certain embodiments, a polypeptide, polynucleotide or composition of the invention is provided in an oral form for administration to a subject in accordance with the methods of the invention.

### Medicaments and Kits

Polypeptides, polynucleotides and/or compositions of the invention may be used in the preparation of medicaments for preventing or treating CLE in a subject.

The subject may be an individual of any mammalian species including, but not limited to, members of the genus ovine, bovine, equine, porcine, feline, canine, primates, and rodents. In one embodiment, the subject is a mammal. Preferably, the mammal is a human suffering from CLE or pre-disposed to developing CLE.

The invention also provides use of a polypeptide, polynucleotide and/or composition of the invention for the prevention or treatment of CLE.

Also provided are kits for the prevention or treatment of CLE. The kits comprise one or more polypeptides, polynucleotides and/or compositions of the invention.

As previously stated, CLE as contemplated herein specifically excludes SLE. Methods for the diagnosis of CLE and SLE are known in the art and referred to above in the section entitled “Therapeutic methods”.

Non-limiting examples of specific forms of CLE that may be treated or prevented using medicaments or kits of the invention include chronic cutaneous lupus erythematosus, subacute cutaneous lupus erythematosus, discoid lupus erythematosus, lupus erythematosus NOS, hypertrophic lupus erythematosus, tumid lupus erythematosus (also known as lupus erythematosus tumidus), lupus panniculitis, lupus profundus, cutaneous lupus mucinosis, acute cutaneous lupus erythematosus, chronic cutaneous lupus erythematosus, drug-induced lupus erythematosus, and intermittent cutaneous lupus erythematosus.

It will be understood that combinations of different polypeptides, polynucleotides and/or compositions of the invention may be included in a kit of the invention or used in the preparation of a medicament of the invention.

Kits of the invention may include other components required to conduct the methods of the invention such as, for example, enzymes, buffers and/or diluents. The kits may comprise one or more means for obtaining a sample from a subject. The kits typically include containers for housing the various components and instructions for using the kit components in the methods of the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

### EXAMPLES

The invention will now be described with reference to specific examples, which should not be construed as in any way limiting.

### Example 1

### Effect of Cpn10 Administration on MRL^{lpr/lpr} Mice

### 1. Materials and Methods

1.1 Activity of Cpn10 in Female MRL^{lpr/lpr} Mice

1.1.1 Rationale

MRL^{lpr/lpr} mice were used for the experiments described herein. In the MRL^{lpr/lpr} mouse a mutation of the Fas gene, by the insertion of early transposable element ETn that causes a striking reduction in Fas mRNA expression, is associated clinically with marked acceleration of lupus-like disease. Cpn10 in two different forms (Ala-Cpn10-Y75K, Gly-Cpn10) and two different concentrations (100 μg/100 μl and 200 μg/100 μl) was tested for its anti-inflammatory and immunosuppressive activity in this model, as compared with mice receiving formulation buffer control injections.

1.1.2 Methods

1.1.2.1 Materials and Methods

MRL^{lpr/lpr} mice 1 (lpr is for lymphoproliferation) aged 6-8 weeks were obtained from the animal facility of HARLAN GmbH and acclimatised for three weeks prior to study initiation. Cpn10 was administered at the dose of 100 μg/100 μl and 200 μg/100 μl every second day by intraperitoneal injections. Formulation buffer (50 mM Tris pH7.6, 150 mM Nacl) used for each formulation was used as control.

1.1.2.2 Test System

Species: Mice

Strain: MRL/MpOlaHsd/lpr (MRL^{lpr/lpr})

Source: HARLAN GmbH (Germany)

Number on Study: Approximately 65 animals (female) were procured from HARLAN GmbH.

Body weight: 25-30 g at initiation of drug treatment.

Age: 11 wks old at the initiation of the drug treatment.

Identification System: Animals were identified with markings on tail. Individual cage cards were affixed to each cage that would display details such as the animal number, the study number, the initiation and termination dates and the experimenters.

Justification for selection: In the MRL^{lpr/lpr} mouse a mutation of the Fas gene, by the insertion of early transposable element ETn causes a striking reduction in Fas mRNA expression and is associated clinically with marked acceleration of the lupus-like disease. The purpose of this study is to evaluate the effect of Cpn10 in a murine model of Lupus (MRL^{lpr/lpr}).

1.1.2.3 Experimental Design

### A. Overview

Each animal received respective compounds in the morning, administered as intraperitoneal injection every other day for 10 weeks (11-22 wks of age). At 22 weeks of age, 2-3 hours, after the first dose of the week, blood samples were collected and animals were sacrificed to obtain tissue samples for histology and morphology.

### B. Animal Receipt, Acclimatization, and Monitoring

Animals judged to be in good health and suitable as test animals were placed in quarantine for at least four weeks. During the acclimatization and biological phase of the study, each animal was observed daily for changes in general appearance and behaviour.

### C. Grouping

Near the end of quarantine/acclimatization period, animals were judged to be suitable for testing. At week 11 of age, animals were grouped as 13 animals per group.

### D. Route of Test Material Administration

The route of Cpn10 administration was by intra-peritoneal injection.

### E. Dosage Levels and Group Assignment

Animals used in the study were divided into groups as outlined in Table 2.

TABLE 2
animal groups.

| | | Dosage | Dose | Sex of | Number of |
|---|---|---|---|---|---|
| Group | Treatment | level | Volume | animal | animals |
| 1 | Formulation Buffer | — | 100 μl | Female | 13 |
| 2 | Ala-Cpn10-Y75K | 100 μg | 100 μl | Female | 13 |
| 3 | Ala-Cpn10-Y75K | 200 μg | 100 μl | Female | 13 |
| 4 | Gly-Cpn10 | 100 μg | 100 μl | Female | 13 |
| 5 | Gly-Cpn10 | 200 μg | 100 μl | Female | 13 |

### F. Formulation of Test Substance

All the test articles (Cpn10 solution and Formulation buffer) were kept frozen until use.

### G. Biological Phase

Weighing of experimental animals: Animals were weighed once per week.

Drug administration: Test articles were thawed on ice and mixed gently before injecting to mice. Drug was administered by intra peritoneal injection every other day.

Collection of blood samples: Blood samples were collected under isoflorane anaesthesia through out the study. Heparanized capillaries were used to collect the blood via retro-orbital bleeding. Plasma was separated by centrifugation at 10,000 rpm for 5 min. To analyse cytokines, IgG and various activity parameters, plasma samples were collected prior to sacrifice the animals after 11 weeks of treatment. Blood was drawn by retro-orbital bleeding using heparinised capillaries three hours after injection. Plasma was separated by centrifugation at 10,000 rpm for 5 min.

Urine samples: Urine samples were collected in week 22 of age.

Clinical signs and mortality record: Clinical signs (skin lesion) were recorded for each group over the study period and mortality was recorded every week through out the study course.

Tissue samples: After ten weeks treatment (week 22 of age) animal groups were sacrificed for tissue collection. Tissue samples for histology were collected and immediately fixed in formalin prior to paraffin fixation. Cryo samples and RNA samples were flash frozen in liquid nitrogen before storing at −70° C. in DEPC-treated Eppendorf tubes.

FACS Analysis: Single cell suspension of splenocytes was obtained from each treatment group. Flow cytometry was performed using the previously characterized method for autoreactive T cells, regulatory T cells and B cells. All antibodies were purchased from BD Pharmingen, including FITC anti-mouse CD3ε APC anti-mouse CD4, PerCP anti-mouse CD8a, PerCP-Cy5.5 anti-mouse CD25, PE anti-mouse CD45R/B220 and PE anti-mouse CD11c. FITC anti-mouse MHCII was purchased from eBiosciences.

### H. Analytical Phase

Biochemical analysis of plasma and urine samples: Urine albumin (Bethyl Laboratories) was estimated using commercially available diagnostic kits. Plasma samples were analysed for cytokines (BD Pharmingen kits for cytokines) and immunoglobulin (Bethyl Laboratories) using ELISA kits.

Histological analysis: Histological analysis was done on paraffin fixed tissue sections. Sections were stained for histology by Periodic Acid Schiff (PAS) stain.

### I. Calculation and Statistical Evaluation

Calculation of mean values: Mean values for each analysis were calculated using MS Excel.

Calculation of significance: Significance for each analysis was determined by using Graph Pad Prism either by ANOVA (Bonferroni's post hoc test) or T test.

### 2. Results

2.1 Systemic Autoimmunity

### A. Survival Rate

In MRL^{lpr/lpr} mice the average median survival of female mice is approximately 22 wks. Cpn10 at doses of 100 μg resulted in prolongation of survival, when compared to Formulation Buffer-treated control mice. Survival for Formulation buffer control mice was around 65% at 22 weeks of age, whereas Ala-Cpn10-Y75K and Gly-Cpn10 administration at 100 μg/100 μl every second day demonstrated survival of more than 90% at 22 weeks of age.

### B. Spleen and Lymph Node Weight

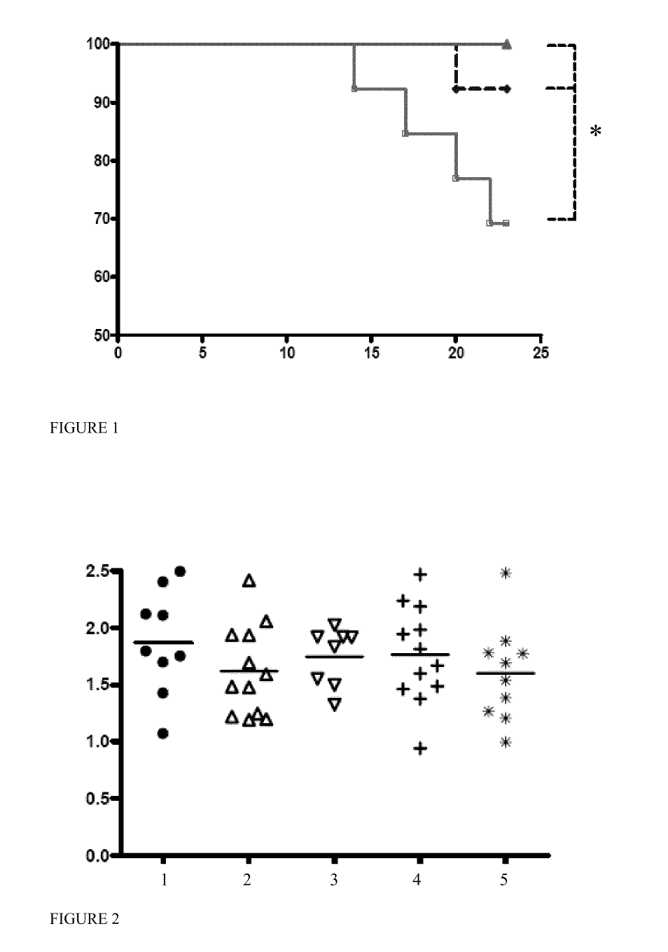

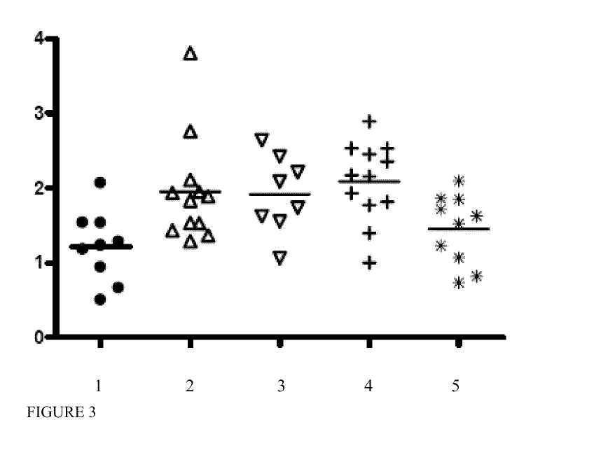

Splenomegaly (FIG. 2) and lymphadenopathy (FIG. 3) are morphological clinical signs for lupus in MRL^{lpr/lpr} mice. Treatment with Ala-Cpn10-Y75K or Gly-Cpn10 did not demonstrate significant effects on these parameters.

### C. Proportion of CD3⁺CD4⁻CD8⁻ T Cells

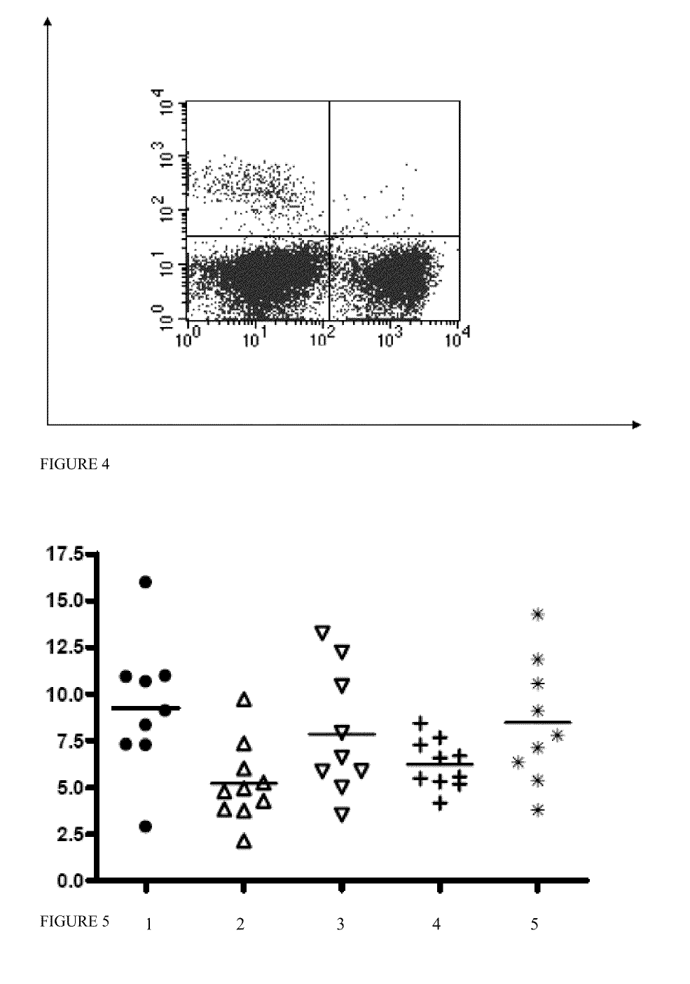

An expansion of CD3⁺CD4⁻CD8⁻ T cells (which are increased in systemic lupus erythematosus patients) was observed in vehicle-treated (control) MRL^{lpr/lpr} mice at week 22. Cpn10 treatment showed a reduction in percentage of CD3⁺CD4⁻CD8⁻ cells (FIGS. 4 and 5) but only with low dose treatment. Loss of activity in high dose treatment has to be explained by further experiments.

### D. Proportion of CD3⁺CD25⁺ T Cells

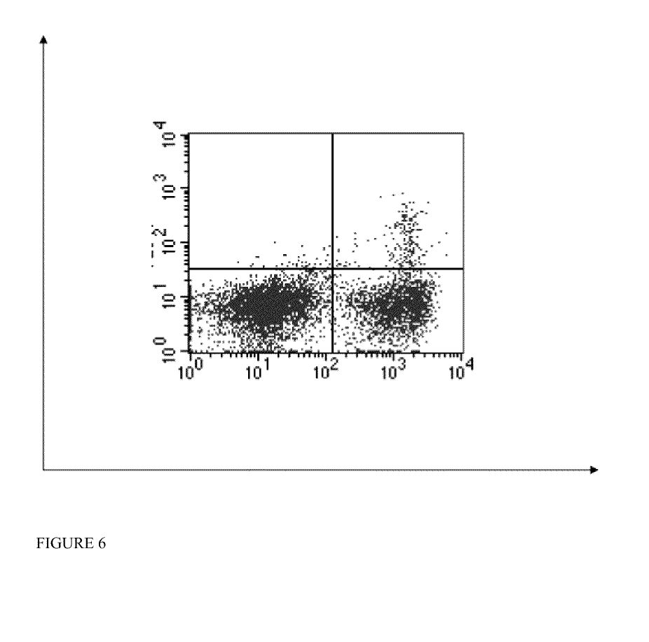

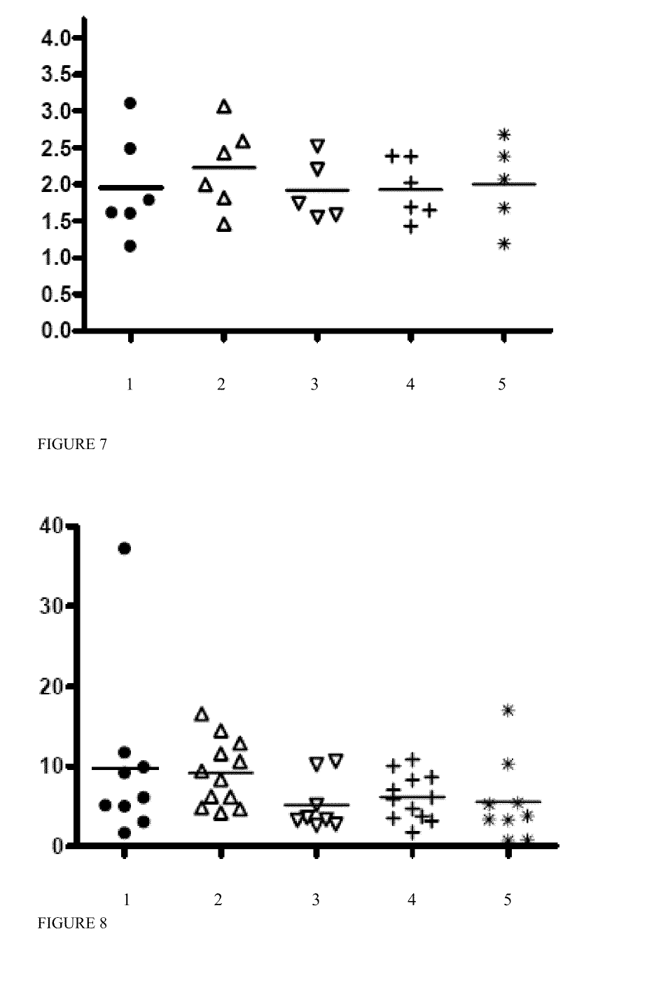

The CD4⁺CD25⁺ regulatory T cells (Tregs) constitute 5-10% of peripheral CD4 T cells in normal naive mice and humans. The direct removal of CD4 Tregs from MRL^{lpr/lpr} mice breaks self-tolerance, leading to the development of autoimmune disease, whereas repopulation of these cells can re-establish self-tolerance and prevent autoimmune disease. This observation indicates that Tregs play a critical role in the maintenance of self-tolerance and the prevention of organ-specific autoimmunity. The depletion of Tregs was observed in vehicle (control) treated mice (FIGS. 6 and 7), however, these were also depleted in Ala-Cpn10-Y75K and Gly-Cpn10-treated groups.

TABLE 3
flow cytometry of spleen cells for Dendritic and B cells activation.

| | Dendritic Cells | | B Cells | |
|---|---|---|---|---|
| | CD11c⁺ MHCII⁺ | | B220⁺ | |
| Group | Mean | SEM | Mean | SEM |
| Formulation Buffer | 1.45 | 0.25 | 46.71 | 3.31 |
| Gly-Cpn10 (100 μg/100 μl) | 2.53 | 0.32 | 37.10 | 2.58 |
| Gly-Cpn10 (200 μg/100 μl) | 1.85 | 0.49 | 46.16 | 2.50 |
| Ala-Cpn10-Y75K (100 μg/100 μl) | 1.35 | 0.08 | 41.78 | 1.94 |
| Ala-Cpn10-Y75K (200 μg/100 μl) | 1.75 | 0.27 | 45.52 | 4.34 |

Treatment with Ala-Cpn10-Y75K or Gly-Cpn10 had no significant effect on either CD11c+MHCII+ cell or B220+cell number (from splenocyte single cell suspension) at week 22 (Table 3).

### E. Serum Parameters

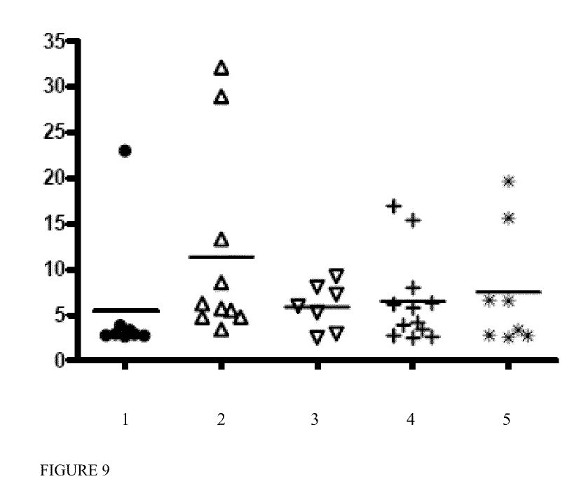

Autoimmunity in MRL^{lpr/lpr} mice is characterised by the production of autoantibodies against multiple nuclear antigens, including dsDNA. Treatment with Ala-Cpn10-Y75K or Gly-Cpn10 did not show any effect on plasma Total IgG (FIG. 8) and plasma ds-DNA specific autoantibody (FIG. 9) levels.

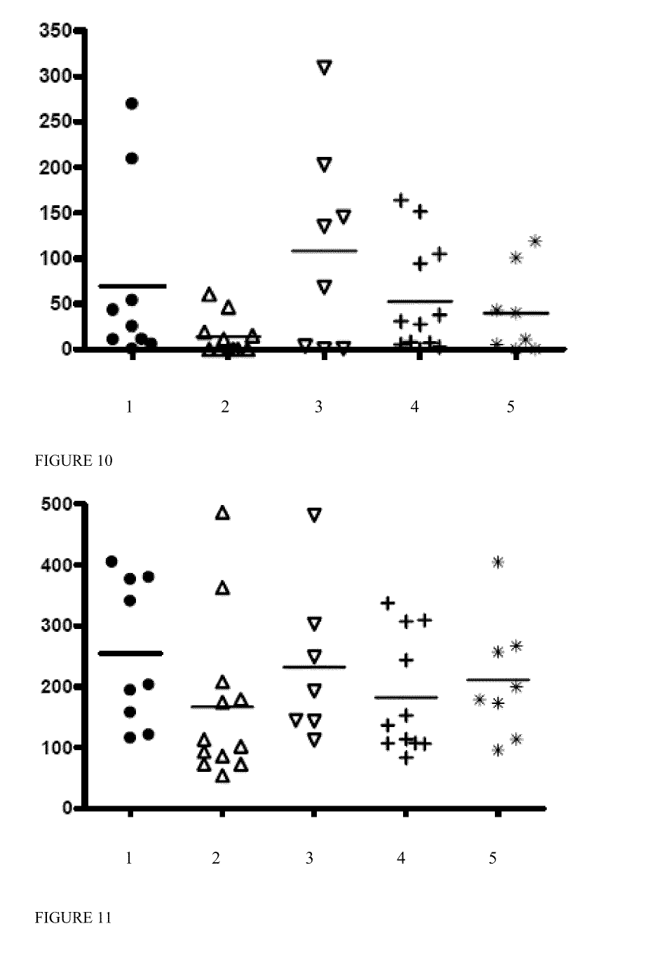

Autoimmunity in MRL^{lpr/lpr} mice is characterised by the increased production of serum cytokines High levels of serum TNF-α (FIG. 11) and IFN-γ (FIG. 10) were observed in 22-week-old MRL^{lpr/lpr} mice. Administration of Ala-Cpn10-Y75K or Gly-Cpn10 had no apparent effect on the levels of these cytokines

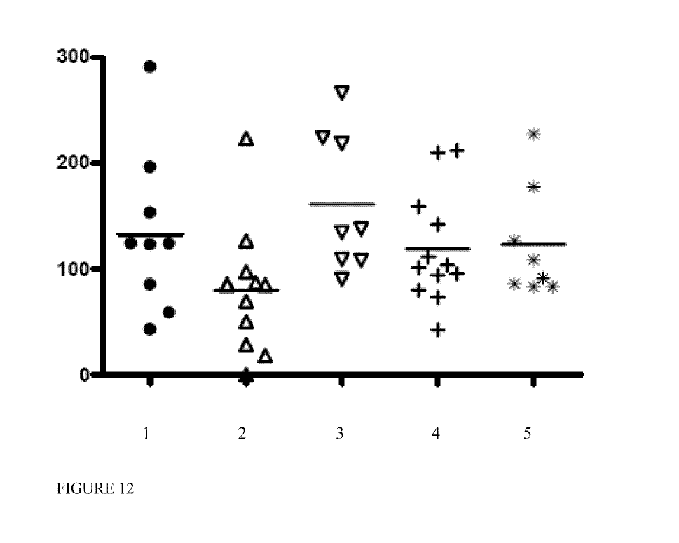

Administration of Ala-Cpn10-Y75K or Gly-Cpn10 also had no apparent effect on plasma IL-10 levels (FIG. 12).

2.2 Renal Parameters

### A. Urine Albumin/Creatinine Ratio

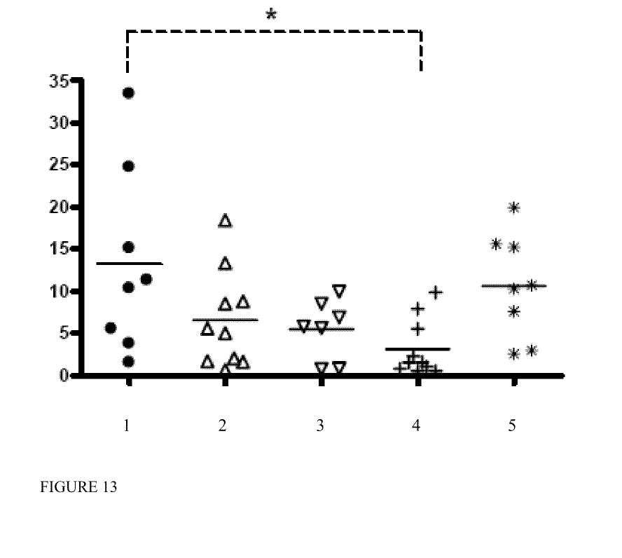

Albumin/creatinine ratio is an important clinical parameter for renal disease. In lupus nephritis it increases with the progression of kidney inflammation. Only the group treated with Ala-Cpn10-Y75K (100 μg/100 μl ) showed significant reduction in albumin/creatinine ratio (FIG. 13). Other treatment groups did not exhibit any effect.

B. Plasma creatinine

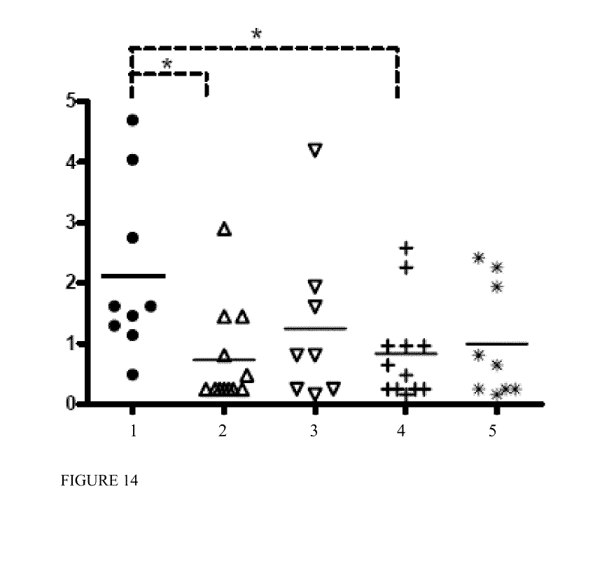

With progression of inflammation there is a loss of filtering ability in the kidney. In MRL^{lpr/lpr} mice, GFR decreases with age. Clearance of plasma creatinine reciprocates the renal functionality. Plasma creatinine levels were measured in mice at week 22. The control group showed elevated plasma creatinine levels indicative of the progressive loss of renal function in this group. Groups treated with Gly-Cpn10 and Ala-Cpn10-Y75K (100 μg/100 μl showed significant reduction in plasma creatinine level indicating restoration of renal function in these groups (FIG. 14).

### C. Renal Histology

Female MRL^{lpr/lpr} mice develop and subsequently die from diffuse proliferative complex glomerulonephritis with striking similarities to diffuse proliferative lupus nephritis in humans. MRL^{lpr/lpr} mice show diffuse proliferative glomerulonephritis characterized by glomerular macrophage infiltration and a mixed periglomerular and interstitial inflammatory cell infiltrate consisting of glomerular and interstitial Mac2-positive macrophages and interstitial CD3-positive lymphocytes. In a therapeutic study design we treated MRL^{lpr/lpr} mice with Ala-Cpn10-Y75K and Gly-Cpn10 at two different doses and formulation buffer (control) from 11 to 22 weeks of age. Renal sections of 22 week old MRL^{lpr/lpr} mice from all groups stained with PAS.

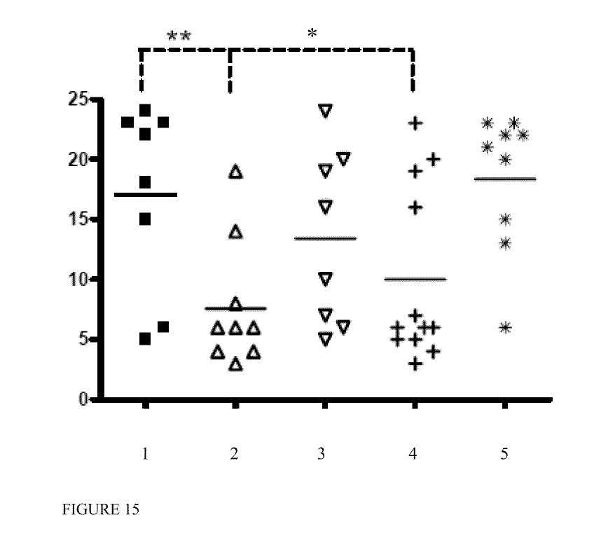

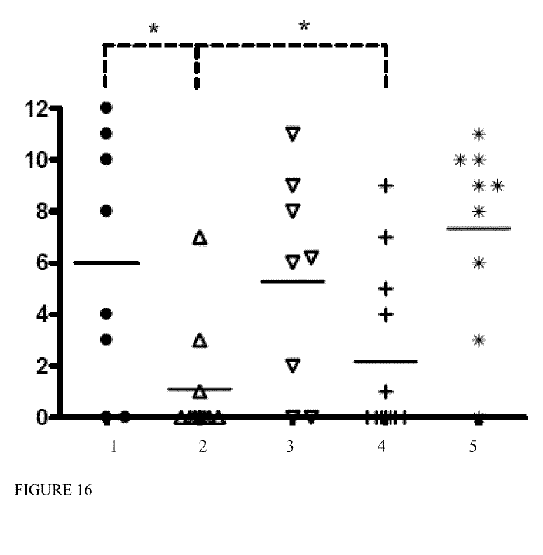

Ala-Cpn10-Y75K and Gly-Cpn10 at 100 μg/μl improved both the activity and chronicity indices of renal inflammation. However, at higher doses both treatments did not show any effect. The activity index (FIG. 15) was defined as the sum of individual scores of the following parameters considered to represent measures of active lupus nephritis: glomerular proliferation, leucocytes exudation, karyorrhexis/fibrinoid necrosis (*2), cellular crescents (*2), hyaline deposits, and interstitial inflammation. The chronicity index (FIG. 16) consisted of the sum of individual scores of the following parameters considered to represent measures of chronic irreversible lupus nephritis: glomerular sclerosis, fibrous crescents, tubular atrophy, and interstitial fibrosis.

2.3 Cutaneous Lupus

Skin lesions are one of the most common manifestations of lupus erythematosus disorders such as discoid lupus erythematosus, subacute lupus erythematosus, lupus erythematosus tumidus and lupus profundus. The analysis of MLR/lpr mice showed a close association between immunoglobulin deposits and the appearance of skin lesions

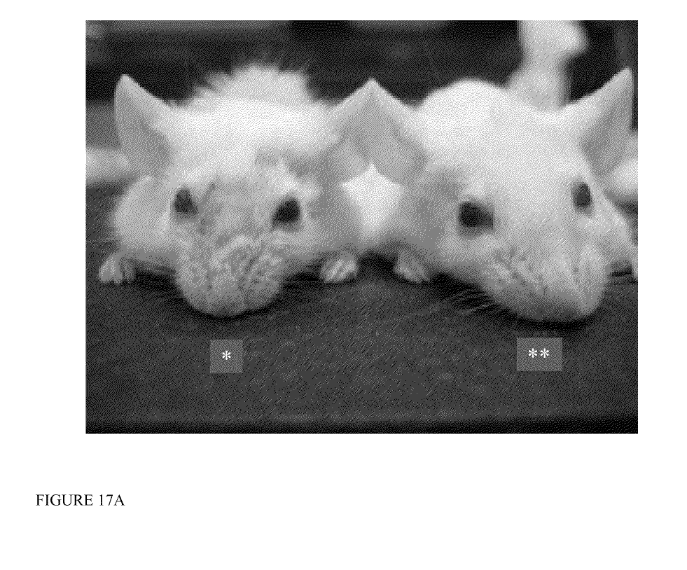

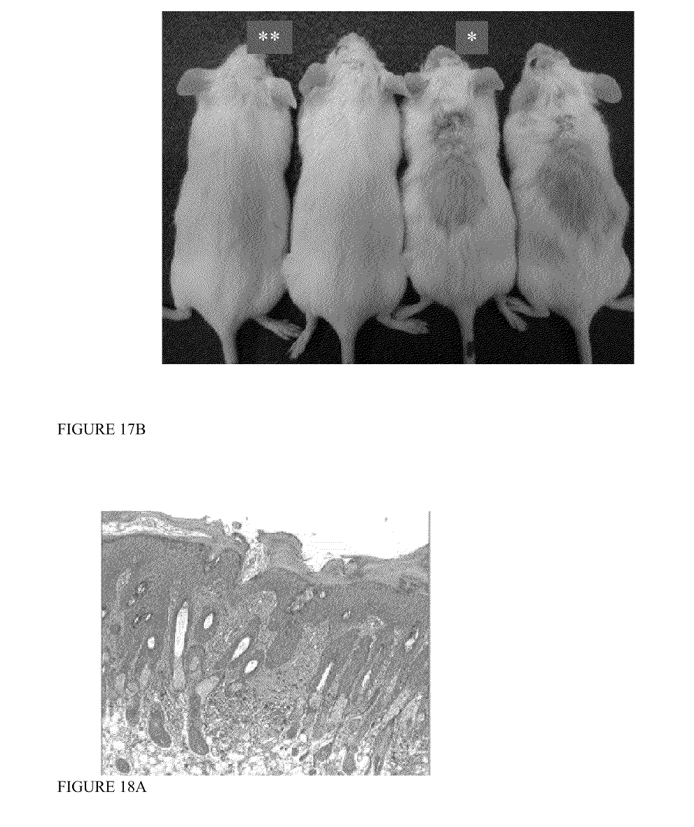

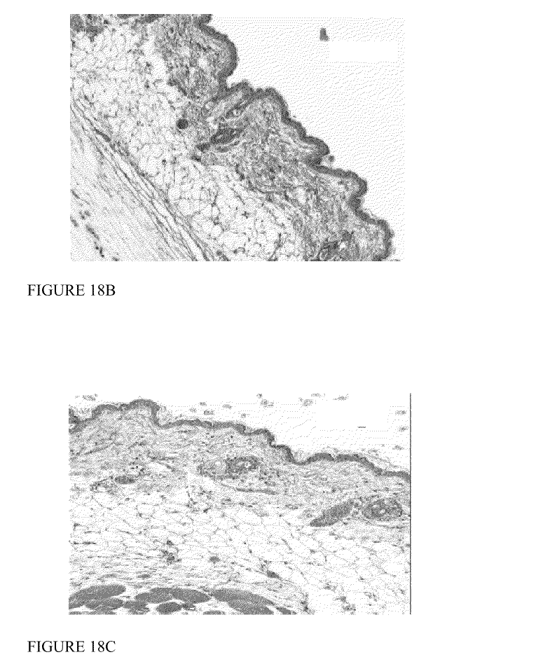

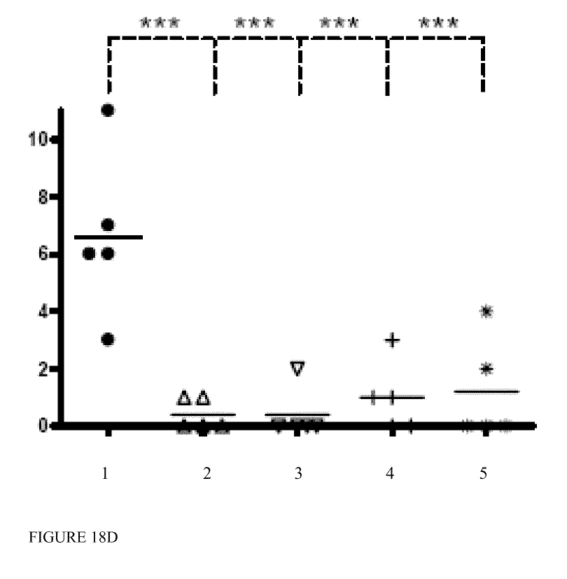

MRL^{lpr/lpr} mice of all groups were regularly checked for cutaneous lupus manifestations, which typically occur at the facial or neck area, and which were not significantly affected in Ala-Cpn10-Y75K or Gly-Cpn10-treated mice. At 22 weeks of age skin tissue was prepared from MRL^{lpr/lpr} mice of all groups and scored semi-quantitatively. Treatments with Cpn10 significantly reduce cutaneous inflammation in MRL^{lpr/lpr} mice (FIG. 17).

1. A method for preventing or treating cutaneous lupus erythematosus in a subject, the method comprising administering a therapeutically effective amount of a chaperonin 10 (Cpn10) polypeptide to the subject.

2. The method according to claim 1, wherein said Cpn10 polypeptide comprises an amino acid sequence as set forth in SEQ ID NO: 1 or SEQ ID NO: 2.

3. The method according to claim 1, wherein said Cpn10 polypeptide is a variant polypeptide sharing at least 70% sequence identity with a wild-type Cpn10 polypeptide, or a fragment thereof.

4. The method according to claim 1 or claim 3, wherein said Cpn10 polypeptide is a variant polypeptide sharing at least 85% sequence identity with a wild-type Cpn10 polypeptide, or a fragment thereof.

5. The method according to claim 3 or claim 4, wherein said variant polypeptide is an N-terminal variant polypeptide comprising an N-terminus extended by at least one additional amino acid residue compared to said wild-type Cpn10 polypeptide.

6. The method according to claim 5, wherein said N-terminal variant polypeptide comprises the amino acid sequence set forth in SEQ ID NO: 4, SEQ ID NO: 7, SEQ ID NO: 97 or SEQ ID NO: 106.

7. The method according to claim 5, wherein said N-terminal variant polypeptide comprises the amino acid sequence set forth in any one of SEQ ID NO: 10, SEQ ID NO: 13, SEQ ID NO: 14, SEQ ID NOs: 17-29, SEQ ID NO: 31, SEQ ID NO: 34, SEQ ID NO: 37, SEQ ID NO: 40, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 62, SEQ ID NO: 65, SEQ ID NO: 68, SEQ ID NO: 71, SEQ ID NO: 73, SEQ ID NO: 76, SEQ ID NO: 77, SEQ ID NO:78, SEQ ID NO: 79, SEQ ID NO: 82, SEQ ID NO: 85, SEQ ID NO: 88, SEQ ID NO: 91, or SEQ ID NO: 93.

8. The method according to claim 3 or claim 4, wherein said variant polypeptide comprises an increased net positive charge in comparison to a wild-type Cpn10 polypeptide.

9. The method according to claim 8, wherein said variant polypeptide comprises the amino acid sequence set forth in any one SEQ ID NO: 97, SEQ ID NO: 100, SEQ ID NO: 103, or SEQ ID NO: 106.

10. The method according to any one of claims 1 to 9, wherein said cutaneous lupus erythematosus is selected from the group consisting of chronic cutaneous lupus erythematosus, subacute cutaneous lupus erythematosus, discoid lupus erythematosus, lupus erythematosus NOS, hypertrophic lupus erythematosus, tumid lupus erythematosus, lupus panniculitis, lupus profundus, cutaneous lupus mucinosis, acute cutaneous lupus erythematosus, chronic cutaneous lupus erythematosus, drug-induced lupus erythematosus, and intermittent cutaneous lupus erythematosus.

11. The method according to any one of claims 1 to 10, wherein said method comprises administering at least one additional agent.

12. The method according to claim 11, wherein said additional agent is one or more of topical steroids, corticosteroids, calcineurin inhibitors, antimalarial drugs, retinoids, methotrexate, thalidomide, ciclosporin, dapsone, gold, clofazamine, cyclophosphamide, and immunoglobulin.

13. Use of a chaperonin 10 (Cpn10) polypeptide in the preparation of a medicament for the prevention or treatment of cutaneous lupus erythematosus.

14. The use according to claim 13, wherein said Cpn10 polypeptide comprises an amino acid sequence as set forth in SEQ ID NO: 1 or SEQ ID NO: 2.

15. The use according to claim 14, wherein said Cpn10 polypeptide is a variant polypeptide sharing at least 70% sequence identity with a wild-type Cpn10 polypeptide, or a fragment thereof.

16. The use according to claim 13 or claim 15, wherein said variant polypeptide is an N-terminal variant polypeptide comprising an N-terminus extended by at least one additional amino acid residue compared to said wild-type Cpn10 polypeptide.

17. The use according to claim 16 wherein said N-terminal variant polypeptide comprises an amino acid sequence as set forth in SEQ ID NO: 4, SEQ ID NO: 7, SEQ ID NO: 10, SEQ ID NO: 13, SEQ ID NO: 14, SEQ ID NOs: 17-29, SEQ ID NO: 31, SEQ ID NO: 34, SEQ ID NO: 37, SEQ ID NO: 40, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 62, SEQ ID NO: 65, SEQ ID NO: 68, SEQ ID NO: 71, SEQ ID NO: 73, SEQ ID NO: 76, SEQ ID NO: 77, SEQ ID NO:78, SEQ ID NO: 79, SEQ ID NO: 82, SEQ ID NO: 85, SEQ ID NO: 88, SEQ ID NO: 91, SEQ ID NO: 93, SEQ ID NO: 97, or SEQ ID NO: 106.

18. The use according to claim 13 or claim 15, wherein said variant polypeptide comprises an increased net positive charge in comparison to a wild-type Cpn10 polypeptide.

19. The use according to claim 18, wherein said variant polypeptide comprises the amino acid sequence set forth in any one SEQ ID NO: 97, SEQ ID NO: 100, SEQ ID NO: 103, or SEQ ID NO: 106.

20. The use according to any one of claims 13 to 19, wherein said cutaneous lupus erythematosus is selected from the group consisting of chronic cutaneous lupus erythematosus, subacute cutaneous lupus erythematosus, discoid lupus erythematosus, lupus erythematosus NOS, hypertrophic lupus erythematosus, tumid lupus erythematosus, lupus panniculitis, lupus profundus, cutaneous lupus mucinosis, acute cutaneous lupus erythematosus, chronic cutaneous lupus erythematosus, drug-induced lupus erythematosus, and intermittent cutaneous lupus erythematosus.

21. A composition comprising a chaperonin 10 (Cpn10) polypeptide and at least one additional agent for the treatment of cutaneous lupus erythematosus.

22. The composition according to claim 21, wherein said additional agent is selected from the group consisting of topical steroids, corticosteroids, calcineurin inhibitors, antimalarial drugs, retinoids, methotrexate, thalidomide, ciclosporin, dapsone, gold, clofazamine, cyclophosphamide, and immunoglobulin.

23. The composition according to claim 21 or claim 22, wherein said Cpn10 polypeptide comprises an amino acid sequence as set forth in SEQ ID NO: 1 or SEQ ID NO: 2.

24. The composition according to claim 21 or claim 22, wherein said Cpn10 polypeptide is a variant polypeptide sharing at least 70% sequence identity with a wild-type Cpn10 polypeptide, or a fragment thereof.

25. The composition according to claim 24, wherein said variant polypeptide comprises an amino acid sequence as set forth in any one of SEQ ID NO: 4, SEQ ID NO: 7, SEQ ID NO: 10, SEQ ID NO: 13, SEQ ID NO: 14, SEQ ID NOs: 17-29, SEQ ID NO: 31, SEQ ID NO: 34, SEQ ID NO: 37, SEQ ID NO: 40, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 62, SEQ ID NO: 65, SEQ ID NO: 68, SEQ ID NO: 71, SEQ ID NO: 73, SEQ ID NO: 76, SEQ ID NO: 77, SEQ ID NO:78, SEQ ID NO: 79, SEQ ID NO: 82, SEQ ID NO: 85, SEQ ID NO: 88, SEQ ID NO: 91, SEQ ID NO: 93, SEQ ID NO: 97, SEQ ID NO: 100, SEQ ID NO: 103, or SEQ ID NO: 106.


SEQUENCE LISTING (108 sequences)

SEQ ID NO: 1 - protein, 102 aa, Homo sapiens
MAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 2 - protein, 101 aa, Artificial Sequence [sequence omitted]

SEQ ID NO: 3 - DNA, 309 bp, Homo sapiens
  Native cDNA of Human Cpn10 (GenBank X75821; GI509780)
atggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 4 - protein, 102 aa, Artificial Sequence
  Description of Artificial Sequence Synthetic polypeptide
AAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 5 - DNA, 312 bp, Artificial Sequence
  Description of Artificial Sequence Synthetic polynucleotide
atggcagcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 6 - DNA, 312 bp, Artificial Sequence
  Description of Artificial Sequence Synthetic polynucleotide
atggcagcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 7 - protein, 102 aa, Artificial Sequence
  Description of Artificial Sequence Synthetic polypeptide
GAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 8 - DNA, 312 bp, Artificial Sequence
  Description of Artificial Sequence Synthetic polynucleotide
atgggtgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 9 - DNA, 312 bp, Artificial Sequence
  Description of Artificial Sequence Synthetic polynucleotide
atgggtgcgggccaggcgtttcgtaaatttctgccgctgtttgatcgtgtgctggttgaacgtagcgcggcggaaaccgtgaccaaaggcggcattatgctgccggaaaaaagccagggcaaagtgctgcaggcgaccgtggttgcggttggcagcggcagcaaaggcaaaggcggcgaaattcagccggtgagcgtgaaagtgggcgataaagtgctgctgccggaatatggcggcaccaaagtggtgctggatgataaagattattttctgttccgcgatggcgatattctgggcaaatatgtggattga

SEQ ID NO: 10 - protein, 102 aa, Artificial Sequence
  Description of Artificial Sequence Synthetic polypeptide
PAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 11 - DNA, 312 bp, Artificial Sequence
  Description of Artificial Sequence Synthetic polynucleotide
atgccggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 12 - DNA, 312 bp, Artificial Sequence
  Description of Artificial Sequence Synthetic polynucleotide
atgccggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 13 - protein, 102 aa, Artificial Sequence
  Description of Artificial Sequence Synthetic polypeptide
HAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 14 - protein, 102 aa, Artificial Sequence
  Description of Artificial Sequence Synthetic polypeptide
SAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 15 - DNA, 312 bp, Artificial Sequence
  Description of Artificial Sequence Synthetic polynucleotide
atgagcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 16 - DNA, 312 bp, Artificial Sequence
  Description of Artificial Sequence Synthetic polynucleotide
atgagcgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 17 - protein, 102 aa, Artificial Sequence
  Description of Artificial Sequence Synthetic polypeptide
VAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 18 - protein, 102 aa, Artificial Sequence
  Description of Artificial Sequence Synthetic polypeptide
LAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 19 - protein, 102 aa, Artificial Sequence
  Description of Artificial Sequence Synthetic polypeptide
IAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 20 - protein, 102 aa, Artificial Sequence
  Description of Artificial Sequence Synthetic polypeptide
FAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 21 - protein, 102 aa, Artificial Sequence
  Description of Artificial Sequence Synthetic polypeptide
YAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 22 - protein, 102 aa, Artificial Sequence
  Description of Artificial Sequence Synthetic polypeptide
WAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 23 - protein, 102 aa, Artificial Sequence
  Description of Artificial Sequence Synthetic polypeptide
CAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 24 - protein, 102 aa, Artificial Sequence
  Description of Artificial Sequence Synthetic polypeptide
TAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 25 - protein, 102 aa, Artificial Sequence
  Description of Artificial Sequence Synthetic polypeptide
DAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 26 - protein, 102 aa, Artificial Sequence
  Description of Artificial Sequence Synthetic polypeptide
NAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 27 - protein, 102 aa, Artificial Sequence
  Description of Artificial Sequence Synthetic polypeptide
EAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 28 - protein, 102 aa, Artificial Sequence
  Description of Artificial Sequence Synthetic polypeptide
QAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 29 - protein, 103 aa, Artificial Sequence
  Description of Artificial Sequence Synthetic polypeptide
MVAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 30 - DNA, 312 bp, Artificial Sequence
  Description of Artificial Sequence Synthetic polynucleotide
atggttgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 31 - protein, 103 aa, Artificial Sequence
  Description of Artificial Sequence Synthetic polypeptide
MLAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 32 - DNA, 312 bp, Artificial Sequence
  Description of Artificial Sequence Synthetic polynucleotide
atgctggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 33 - DNA, 312 bp, Artificial Sequence
  Description of Artificial Sequence Synthetic polynucleotide
atgctggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 34 - protein, 103 aa, Artificial Sequence
  Description of Artificial Sequence Synthetic polypeptide
MIAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 35 - DNA, 312 bp, Artificial Sequence
  Description of Artificial Sequence Synthetic polynucleotide
atgattgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 36 - DNA, 312 bp, Artificial Sequence
  Description of Artificial Sequence Synthetic polynucleotide
atgattgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 37 - protein, 103 aa, Artificial Sequence
  Description of Artificial Sequence Synthetic polypeptide
MHAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 38 - DNA, 312 bp, Artificial Sequence
  Description of Artificial Sequence Synthetic polynucleotide
atgcatgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 39 - DNA, 312 bp, Artificial Sequence
  Description of Artificial Sequence Synthetic polynucleotide
atgcatgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 40 - protein, 103 aa, Artificial Sequence
  Description of Artificial Sequence Synthetic polypeptide
MFAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 41 - DNA, 312 bp, Artificial Sequence
  Description of Artificial Sequence Synthetic polynucleotide
atgttcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 42 - protein, 103 aa, Artificial Sequence
  Description of Artificial Sequence Synthetic polypeptide
MYAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 43 - DNA, 312 bp, Artificial Sequence
  Description of Artificial Sequence Synthetic polynucleotide
atgtatgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 44 - DNA, 312 bp, Artificial Sequence
  Description of Artificial Sequence Synthetic polynucleotide
atgtatgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 45 - protein, 103 aa, Artificial Sequence
  Description of Artificial Sequence Synthetic polypeptide
MWAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 46 - DNA, 312 bp, Artificial Sequence
  Description of Artificial Sequence Synthetic polynucleotide
atgtgggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 47 - DNA, 312 bp, Artificial Sequence
  Description of Artificial Sequence Synthetic polynucleotide
atgtgggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 48 - protein, 103 aa, Artificial Sequence
  Description of Artificial Sequence Synthetic polypeptide
MCAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 49 - DNA, 312 bp, Artificial Sequence
  Description of Artificial Sequence Synthetic polynucleotide
atgtgcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 50 - DNA, 312 bp, Artificial Sequence
  Description of Artificial Sequence Synthetic polynucleotide
atgtgcgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 51 - protein, 103 aa, Artificial Sequence
  Description of Artificial Sequence Synthetic polypeptide
MTAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 52 - DNA, 312 bp, Artificial Sequence
  Description of Artificial Sequence Synthetic polynucleotide
atgaccgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 53 - DNA, 312 bp, Artificial Sequence
  Description of Artificial Sequence Synthetic polynucleotide
atgaccgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 54 - protein, 103 aa, Artificial Sequence
  Description of Artificial Sequence Synthetic polypeptide
MDAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 55 - DNA, 312 bp, Artificial Sequence
  Description of Artificial Sequence Synthetic polynucleotide
atggatgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 56 - DNA, 312 bp, Artificial Sequence
  Description of Artificial Sequence Synthetic polynucleotide
atggatgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 57 - protein, 103 aa, Artificial Sequence
  Description of Artificial Sequence Synthetic polypeptide
MNAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 58 - DNA, 312 bp, Artificial Sequence
  Description of Artificial Sequence Synthetic polynucleotide
atgaacgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 59 - DNA, 312 bp, Artificial Sequence
  Description of Artificial Sequence Synthetic polynucleotide
atgaacgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 60 - protein, 103 aa, Artificial Sequence
  Description of Artificial Sequence Synthetic polypeptide
MEAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 61 - DNA, 312 bp, Artificial Sequence
  Description of Artificial Sequence Synthetic polynucleotide
atggaagcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 62 - protein, 103 aa, Artificial Sequence
  Description of Artificial Sequence Synthetic polypeptide
MQAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 63 - DNA, 312 bp, Artificial Sequence
  Description of Artificial Sequence Synthetic polynucleotide
atgcaggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 64 - DNA, 312 bp, Artificial Sequence
  Description of Artificial Sequence Synthetic polynucleotide
atgcaggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 65 - protein, 103 aa, Artificial Sequence
  Description of Artificial Sequence Synthetic polypeptide
AAAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 66 - DNA, 315 bp, Artificial Sequence
  Description of Artificial Sequence Synthetic polynucleotide
atggcagcggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 67 - DNA, 315 bp, Artificial Sequence
  Description of Artificial Sequence Synthetic polynucleotide
atggcagcggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 68 - protein, 103 aa, Artificial Sequence
  Description of Artificial Sequence Synthetic polypeptide
GGAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 69 - DNA, 315 bp, Artificial Sequence
  Description of Artificial Sequence Synthetic polynucleotide
atgggcggtgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 70 - DNA, 315 bp, Artificial Sequence
  Description of Artificial Sequence Synthetic polynucleotide
atgggcggtgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 71 - protein, 104 aa, Artificial Sequence
  Description of Artificial Sequence Synthetic polypeptide
GSMAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 72 - DNA, 318 bp, Artificial Sequence
  Description of Artificial Sequence Synthetic polynucleotide
atgggaagtatggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 73 - protein, 104 aa, Artificial Sequence
  Description of Artificial Sequence Synthetic polypeptide
AAAAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 74 - DNA, 318 bp, Artificial Sequence
  Description of Artificial Sequence Synthetic polynucleotide
atggcggcagcggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 75 - DNA, 318 bp, Artificial Sequence
  Description of Artificial Sequence Synthetic polynucleotide
atggcggcagcggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 76 - protein, 103 aa, Artificial Sequence
  Description of Artificial Sequence Synthetic polypeptide
QSAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 77 - protein, 104 aa, Artificial Sequence
  Description of Artificial Sequence Synthetic polypeptide
QSMAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 78 - protein, 103 aa, Artificial Sequence
  Description of Artificial Sequence Synthetic polypeptide
MMAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 79 - protein, 104 aa, Artificial Sequence
  Description of Artificial Sequence Synthetic polypeptide
STGAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 80 - DNA, 318 bp, Artificial Sequence
  Description of Artificial Sequence Synthetic polynucleotide
atgagcaccggcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 81 - DNA, 318 bp, Artificial Sequence
  Description of Artificial Sequence Synthetic polynucleotide
atgagcaccggcgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 82 - protein, 103 aa, Artificial Sequence
  Description of Artificial Sequence Synthetic polypeptide
PGAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 83 - DNA, 315 bp, Artificial Sequence
  Description of Artificial Sequence Synthetic polynucleotide
atgccgggcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 84 - DNA, 315 bp, Artificial Sequence
  Description of Artificial Sequence Synthetic polynucleotide
atgccgggcgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 85 - protein, 104 aa, Artificial Sequence
  Description of Artificial Sequence Synthetic polypeptide
PTGAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 86 - DNA, 317 bp, Artificial Sequence
  Description of Artificial Sequence Synthetic polynucleotide
atgccgaccggcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactg

SEQ ID NO: 87 - DNA, 318 bp, Artificial Sequence
  Description of Artificial Sequence Synthetic polynucleotide
atgccgaccggcgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 88 - protein, 104 aa, Artificial Sequence
  Description of Artificial Sequence Synthetic polypeptide
MQSAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 89 - DNA, 315 bp, Artificial Sequence
  Description of Artificial Sequence Synthetic polynucleotide
atgcagagcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 90 - DNA, 315 bp, Artificial Sequence
  Description of Artificial Sequence Synthetic polynucleotide
atgcagagcgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 91 - protein, 105 aa, Artificial Sequence
  Description of Artificial Sequence Synthetic polypeptide
MQSMAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 92 - DNA, 318 bp, Artificial Sequence
  Description of Artificial Sequence Synthetic polynucleotide
atgcagagcatggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 93 - DNA, 318 bp, Artificial Sequence
  Description of Artificial Sequence Synthetic polynucleotide
atgcagagcatggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 94 - protein, 103 aa, Artificial Sequence
  Description of Artificial Sequence Synthetic polypeptide
SGAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 95 - DNA, 315 bp, Artificial Sequence
  Description of Artificial Sequence Synthetic polynucleotide
atgagcggcgcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 96 - DNA, 315 bp, Artificial Sequence
  Description of Artificial Sequence Synthetic polynucleotide
atgagcggcgcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaatatggtggcaccaaagtagttctagatgacaaggattatttcctatttcgtgatggtgacattcttggcaagtacgtagactaa

SEQ ID NO: 97 - protein, 102 aa, Artificial Sequence
  Description of Artificial Sequence Synthetic polypeptide
AAGQAFRKFLPLFDRVLVERSAAETVTKGGIMLPEKSQGKVLQATVVAVGSGSKGKGGEIQPVSVKVGDKVLLPEKGGTKVVLDDKDYFLFRDGDILGKYVD

SEQ ID NO: 98 - DNA, 312 bp, Artificial Sequence
  Description of Artificial Sequence Synthetic polynucleotide
atggcagcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaaaaaggaggcaccaaagtagttctagatgacaaggattatttcctatttagagatggtgacattcttggaaagtacgtagactga

SEQ ID NO: 99 - DNA, 312 bp, Artificial Sequence
  Description of Artificial Sequence Synthetic polynucleotide
atggcagcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaaaaaggtggcaccaaagtagttctagatgacaaggattatttcctatttcgtgatggtgacattcttggcaagtacgtagactaa

SEQ ID NO: 100 - protein, 101 aa, Artificial Sequence
  Description of Artificial Sequence Synthetic polypeptide
AGQAFRKFLPLFDRVLVERSAAETVTKGGIMLPEKSQGKVLQATVVAVGSGSKGKGGEIQPVSVKVGDKVLLPEKGGTKVVLDDKDYFLFRDGDILGKYVD

SEQ ID NO: 101 - DNA, 309 bp, Artificial Sequence
  Description of Artificial Sequence Synthetic polynucleotide
atggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaaaaaggaggcaccaaagtagttctagatgacaaggattatttcctatttagagatggtgacattcttggaaagtacgtagactga

SEQ ID NO: 102 - DNA, 309 bp, Artificial Sequence
  Description of Artificial Sequence Synthetic polynucleotide
atggcaggccaagcgtttcgcaagtttcttccactctttgaccgtgtattggttgaacgcagtgctgctgaaactgtaaccaaaggtggcattatgcttccagaaaaatctcaaggcaaagtattgcaagcaacagtagtcgctgttggctcgggttctaaaggtaagggtggcgagattcaaccagttagcgtgaaagttggcgataaagttcttctcccagaaaaaggtggcaccaaagtagttctagatgacaaggattatttcctatttcgtgatggtgacattcttggcaagtacgtagactaa

SEQ ID NO: 103 - protein, 102 aa, Artificial Sequence
  Description of Artificial Sequence Synthetic polypeptide
MAGQAFRKFLPLFDRVLVERSAAETVTKGGIMLPEKSQGKVLQATVVAVGSGSKGKGGEIQPVSVKVGDKVLLPEKGGTKVVLDDKDYFLFRDGDILGKYVD

SEQ ID NO: 104 - DNA, 309 bp, Artificial Sequence; identical to SEQ ID NO: 101
  Description of Artificial Sequence Synthetic polynucleotide

SEQ ID NO: 105 - DNA, 309 bp, Artificial Sequence; identical to SEQ ID NO: 102
  Description of Artificial Sequence Synthetic polynucleotide

SEQ ID NO: 106 - protein, 103 aa, Artificial Sequence
  Description of Artificial Sequence Synthetic polypeptide
MAAGQAFRKFLPLFDRVLVERSAAETVTKGGIMLPEKSQGKVLQATVVAVGSGSKGKGGEIQPVSVKVGDKVLLPEKGGTKVVLDDKDYFLFRDGDILGKYVD

SEQ ID NO: 107 - DNA, 312 bp, Artificial Sequence; identical to SEQ ID NO: 98
  Description of Artificial Sequence Synthetic polynucleotide

SEQ ID NO: 108 - DNA, 312 bp, Artificial Sequence; identical to SEQ ID NO: 99
  Description of Artificial Sequence Synthetic polynucleotide